(19) United States
(12) Patent Application Publication
(10) Pub. No.: US 2006/0217572 A1
(43) Pub. Date: Sep. 28, 2006

(54) Aromatic amine derivative and organic electroluminescence device employing the same

(75) Inventors: Masahiro Kawamura, Chiba (JP); Nobuhiro Yabunouchi, Chiba (JP); Chishio Hosokawa, Chiba (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Idemitsu Kosan Co., Ltd., Chiyoda-ku (JP)

(21) Appl. No.: 11/362,159

(22) Filed: Feb. 27, 2006

(30) Foreign Application Priority Data
Mar. 25, 2005 (JP) 2005-089470

(51) Int. Cl. H01J 1/62; H01J 63/04; C07C 211/54

(52) U.S. Cl. 564/427; 564/434; 313/504

(57) ABSTRACT

To provide an organic electroluminescent device showing various luminescent color tones and having high heat resistance, a long lifetime, high emission luminance, and high emission efficiency, in particular, an organic electroluminescent device capable of preventing the attenuation of emission luminance in association with the driving of the device. Provided is an organic electroluminescent device including: an aromatic amine compound having a specific structure; a cathode; an anode; and one or multiple organic thin film layers having at least a light-emitting layer, the one or multiple organic thin film layers being interposed between the cathode and the anode, in which at least one layer of the one or multiple organic thin film layers contains the aromatic amine compound alone or as a component of a mixture.

### TECHNICAL FIELD

The present invention relates to an aromatic amine derivative and an organic electroluminescent device using the same. More specifically, the present invention relates to an organic electroluminescent device showing various luminescent color tones and having high heat resistance, a long lifetime, high emission luminance, and high emission efficiency and to a novel aromatic amine derivative for realizing the organic electroluminescent device.

### BACKGROUND ART

An organic electroluminescent device (hereinafter, electroluminescence sometimes abbreviated as “EL”) is a spontaneous light-emitting device which utilizes the principle that a fluorescent substance emits light by energy of recombination of holes injected from an anode and electrons injected from a cathode when an electric field is applied. Since an organic EL device of the laminate type driven under a low electric voltage was reported by C. W. Tang of Eastman Kodak Company (C. W. Tang and S. A. Vanslyke, Applied Physics Letters, Volume 51, Pages 913, 1987), many studies have been conducted on organic EL devices using organic materials as the constituting materials. Tang et al. used tris(8-quinolinolato)aluminum for the light-emitting layer and a triphenyldiamine derivative for the hole-transporting layer. Advantages of the laminate structure are that the efficiency of hole injection into the light-emitting layer can be increased, that the efficiency of forming excitons which are formed by blocking and recombining electrons injected from the cathode can be increased, and that exciton formed within the light-emitting layer can be enclosed. As the structure of the organic EL device, a two-layered structure having a hole-transporting (injecting) layer and an electron-transporting and light-emitting layer and a three-layered structure having a hole-transporting (injecting) layer, a light-emitting layer and an electron-transporting (injecting) layer are well known. To increase the efficiency of recombination of injected holes and electrons in the devices of the laminate type, the structure of the device and the process for forming the device have been studied.

Typically, an aromatic diamine derivative described in Patent Document 1 and an aromatic fused ring diamine derivative described in Patent Document 2 are know as a hole-transporting material to be used in an organic EL device.

In general, when an organic EL device is driven or stored in an environment of a high temperature, adverse effects such as a change in the luminescent color, a decrease in emission efficiency, an increase in the voltage for driving and a decrease in the lifetime of light emission arise. To prevent the adverse effects, it has been necessary that the glass transition temperature (Tg) of the hole-transporting material be elevated. For example, a tetramer of an aromatic amine used for the hole-transporting material having high Tg as disclosed in Patent Documents 3, 4 and 5 are known.

However, each of those materials has a high evaporation temperature, and an organic EL device formed of each of those materials shows the abrupt attenuation of emission luminance in association with the driving of the device. The attenuation is remarkable in the case of a blue light-emitting device.

Patent Document 1: U.S. Pat. No. 4,720,432

Patent Document 2: U.S. Pat. No. 5,061,569

Patent Document 3: JP-B-3220950

Patent Document 4: JP-B-3194657

Patent Document 5: JP-B-3180802

### DISCLOSURE OF THE INVENTION

### Problems to be Solved by the Invention

The present invention has been made with a view to solving the above problems, and an object of the present invention is to provide an organic EL device showing various luminescent color tones and having high heat resistance, a long lifetime, high emission luminance, and high emission efficiency, in particular, an organic EL device capable of preventing the attenuation of emission luminance in association with the driving of the organic EL device, and a novel aromatic amine compound for realizing the organic EL device.

### MEANS FOR SOLVING THE PROBLEMS

The inventors of the present invention have made extensive studies with a view to achieving the above object. As a result, they have found that an aromatic amine derivative represented by the following general formula (I) achieves the above object, thereby completing the present invention.

That is, the present invention provides an aromatic amine derivative having a specific structure represented by the following general formula (I).

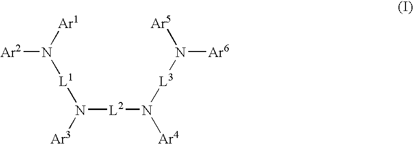

The present invention also provide an organic EL device including: a cathode; an anode; and one or multiple organic thin film layers having at least a light-emitting layer, the one or multiple organic thin film layers being interposed between the cathode and the anode, in which at least one layer of the one or multiple organic thin film layers contains an aromatic amine derivative represented by the general formula (I) alone or as a component of a mixture. With the organic EL device, the above object has been achieved.

### EFFECT OF THE INVENTION

An organic EL device using the aromatic amine derivative of the present invention shows various luminescent color tones, and has high heat resistance. In particular, when the aromatic amine derivative of the present invention is used as a hole-injecting/transporting material, the organic EL device has a long lifetime, high emission luminance, and high emission efficiency, and, in particular, the attenuation of the emission luminance of the organic EL device can be prevented.

### BEST MODE FOR CARRYING OUT THE INVENTION

According to a first invention of the present invention, there is provided an aromatic amine derivative represented by the following general formula (I).

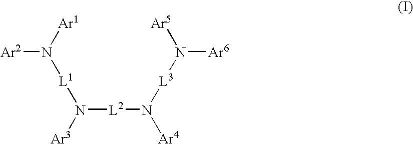

In the general formula (1), Ar¹ to Ar⁶ each independently represent a substituted or unsubstituted aryl group having 6 to 20 nuclear atoms. Examples of the aryl group include a phenyl group, a1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenylyl group, a 4″-t-butyl-p-terphenyl-4-yl group, and a fluorenyl group.

Of those, a phenyl group, a naphthyl group, a biphenyl group, an anthryl group, a phenanthryl group, a pyrenyl group, a chrysenyl group, and a fluorenyl groups are preferable. Of those, a phenyl group and a naphthyl group are most preferable.

L¹ to L³ in the general formula (I) each independently represent a linking group represented by the following general formula (II).

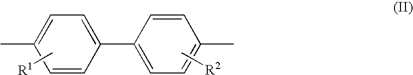

In the general formula (II), R¹ and R² each independently represent a hydrogen atom, a substituted or unsubstituted alkyl group having 1 to 6 carbon atoms, or a substituted or unsubstituted aryl group having 6 to 20 nuclear carbon atoms.

Examples of a substituted or unsubstituted alkyl group having 1 to 6 carbon atoms represented by R¹ or R² in the general formula (II) include a methyl group, an ethyl group, an n-propyl group, an isopropyl group, an n-butyl group, an s-butyl group, a t-butyl group, an n-pentyl group, a cyclopentyl group, an n-hexyl group, and a cyclohexyl group.

Specific examples of a substituted or unsubstituted aryl group having 6 to 20 nuclear carbon atoms represented by R¹ or R² in the general formula (II) include the same specific examples as those of Ar¹ to Ar⁶ in the general formula (I).

R¹ and R² in the general formula (II) may be coupled with each other to form a saturated or unsaturated ring.

It should be noted that Ar¹ to Ar⁶ in the general formula (I) satisfy one of the following conditions (a) to (c)

(a) At least two of Ar¹ to Ar³ each independently represent a substituted or unsubstituted fused aromatic ring having 10 to 20 nuclear carbon atoms.

(b) At least one of Ar³ and Ar⁴ represents a substituted or unsubstituted fused aromatic ring having 10 to 20 nuclear carbon atoms.

(c) Only one of Ar¹, Ar², Ar⁵, and Ar⁶ represents a substituted or unsubstituted fused aromatic ring having 10 to 20 nuclear carbon atoms.

In the aromatic amine derivative of the present invention, at least two of Ar¹ to Ar³ in the general formula (I) preferably each independently represent a substituted or unsubstituted fused aromatic ring having 10 to 20 nuclear carbon atoms.

In the aromatic amine derivative of the present invention, at least one of Ar³ and Ar⁴ in the general formula (I) preferably represents a substituted or unsubstituted fused aromatic ring having 10 to 20 nuclear carbon atoms.

In the aromatic amine derivative of the present invention, only one of Ar¹, Ar², Ar⁵, and Ar⁶ in the general formula (I) preferably represents a substituted or unsubstituted fused aromatic ring having 10 to 20 nuclear carbon atoms.

Examples of a fused aromatic ring having 10 to 20 nuclear carbon atoms represented by any one of Ar¹ to Ar 6include a naphthyl group, a phenanthryl group, an anthryl group, a pyrenyl group, a chrysenyl group, an acenaphthyl group, and a fluorenyl group. Of those, a naphthyl group and a phenanthryl group are preferable.

According to a second invention of the present invention, there is provided an aromatic amine derivative represented by the following general formula (I′).

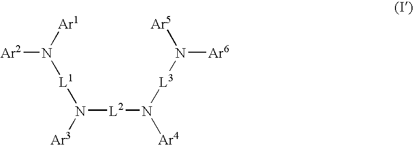

In the formula, Ar¹ to Ar⁶ each independently represent a substituted or unsubstituted aryl group having 6 to 20 nuclear atoms. Specific examples of such group include the same groups as those exemplified for Ar¹ to Ar⁶ in the general formula (I). At least one of Ar¹ to Ar⁶ represents a substituted or unsubstituted 2-naphthyl group.

L¹ to L³ each independently represent a linking group represented by the following general formula (II′).

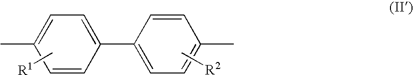

In the formula, R¹ and R² each independently represent a hydrogen atom, a substituted or unsubstituted alkyl group having 1 to 6 carbon atoms, or a substituted or unsubstituted aryl group having 6 to 20 nuclear carbon atoms. Specific examples of such group include the same groups as those exemplified for R¹ and R² in the general formula (II). R¹ and R² may be coupled with each other to form a saturated or unsaturated ring.

In aromatic amine derivative of the present invention, at least one of Ar³ and Ar⁴ in the general formula (I′) preferably represents a substituted or unsubstituted 2-naphthyl group.

In the aromatic amine derivative of the present invention, at least one of Ar¹ and Ar⁵ in the general formula (I′) preferably represents a substituted or unsubstituted 2-naphthyl group.

In the aromatic amine derivative of the present invention, Ar³ and Ar⁴ in the general formula (I′) preferably each represent a substituted or unsubstituted 2-naphthyl group.

In the aromatic amine derivative of the present invention, Ar¹ and Ar⁵ in the general formula (I′) preferably each represent a substituted or unsubstituted 2-naphthyl group.

In the aromatic amine derivative of the present invention, Ar² to Ar⁴ and Ar⁶ in the general formula (I′) preferably each independently represent a substituted or unsubstituted aryl group having 6 to 20 nuclear atoms.

In the aromatic amine derivative of the present invention, L¹ to L³ in the general formula (I) are preferably each independently selected from the linking group consisting of the following general formulae (III-1) to (III-4).

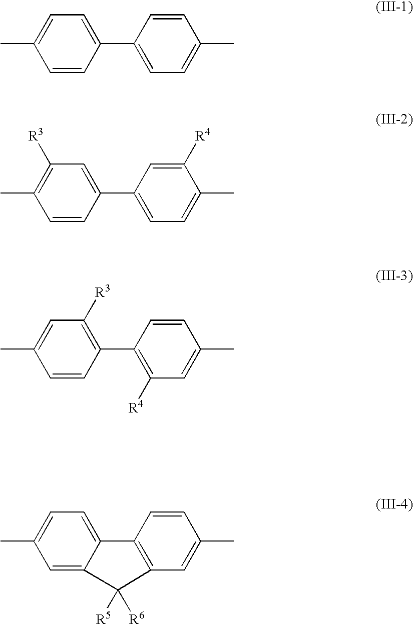

R³ to R⁶ in the general formulae (III-1) to (III-4) each independently represent a substituted or unsubstituted alkyl group having 1 to 6 carbon atoms, or a substituted or unsubstituted aryl group having 6 to 20 nuclear carbon atoms. Specific examples of such group include the same groups as those exemplified for R¹ and R² in the general formula (II). R⁵ and R⁶ may be coupled with each other to form a saturated or unsaturated ring.

The aromatic amine derivative of the present invention represented by the general formula (I) is preferably a material for organic EL.

The aryl groups having 6 to 20 nuclear carbon atoms, the alkyl groups having 1 to 6 carbon atoms, and the fused aromatic ring having 10 to 20 nuclear carbon atoms may be further substituted with substituents including: alkyl groups (such as a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, and a 2-norbornyl group); alkoxy groups having 1 to 6 carbon atoms (such as an ethoxy group, a methoxy group, i-propoxy group, a n-propoxy group, s-butoxy group, a t-butoxy group, a pentoxy group, a hexyloxy group, a cyclopentoxy group, and a cyclohexyloxyl group); aryl groups having 5 to 40 nuclear atoms; an amino group substituted with an aryl group having 5 to 40 nuclear atoms; an ester group substituted with an aryl group having 5 to 40 nuclear atoms; an ester group substituted with an alkyl group having 1 to 6 carbon atoms; a cyano group; a nitro group; and a halogen atom.

Specific examples of the compounds represented by the general formulae (I) and (I′) are shown in the following. However, the compounds are not limited to those shown as the examples. Further, in the figure, Me represents a methyl group.

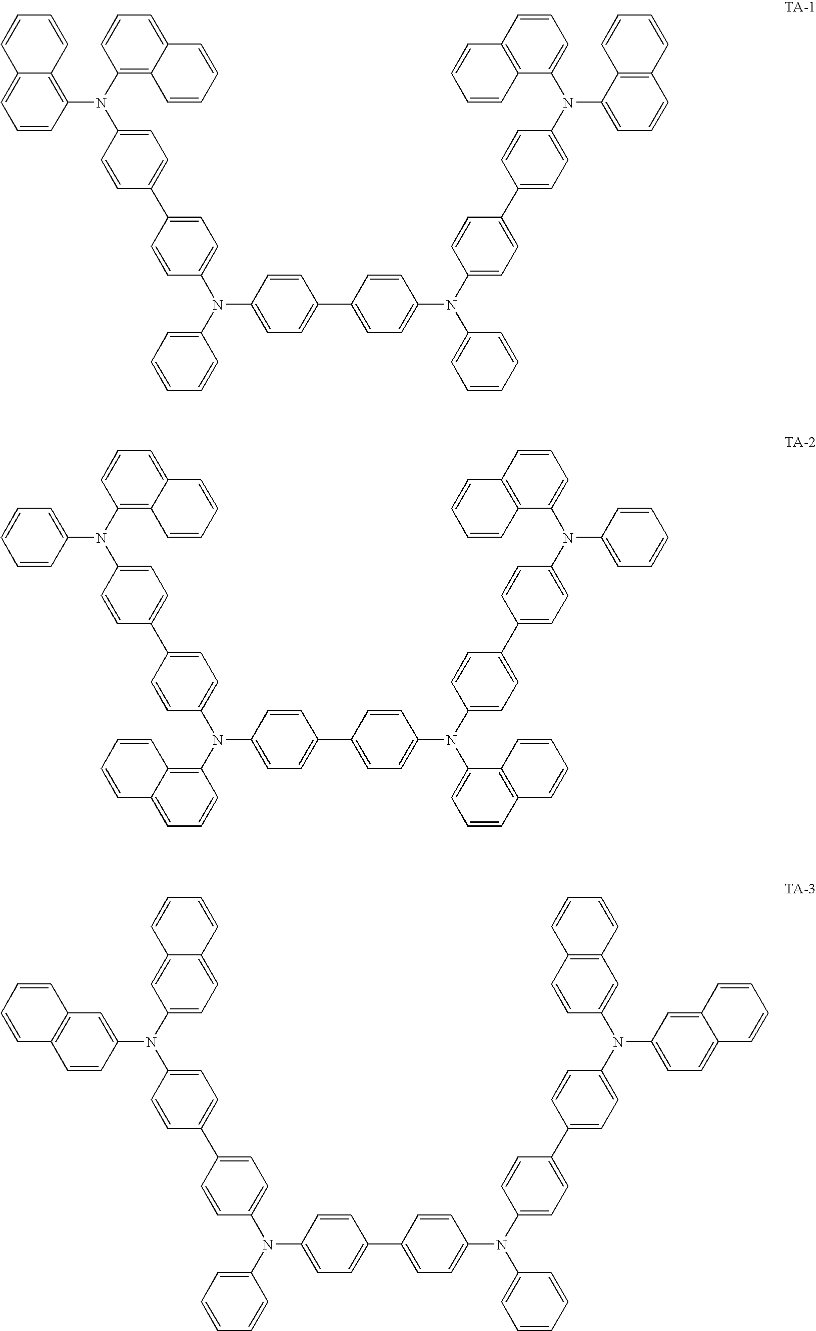

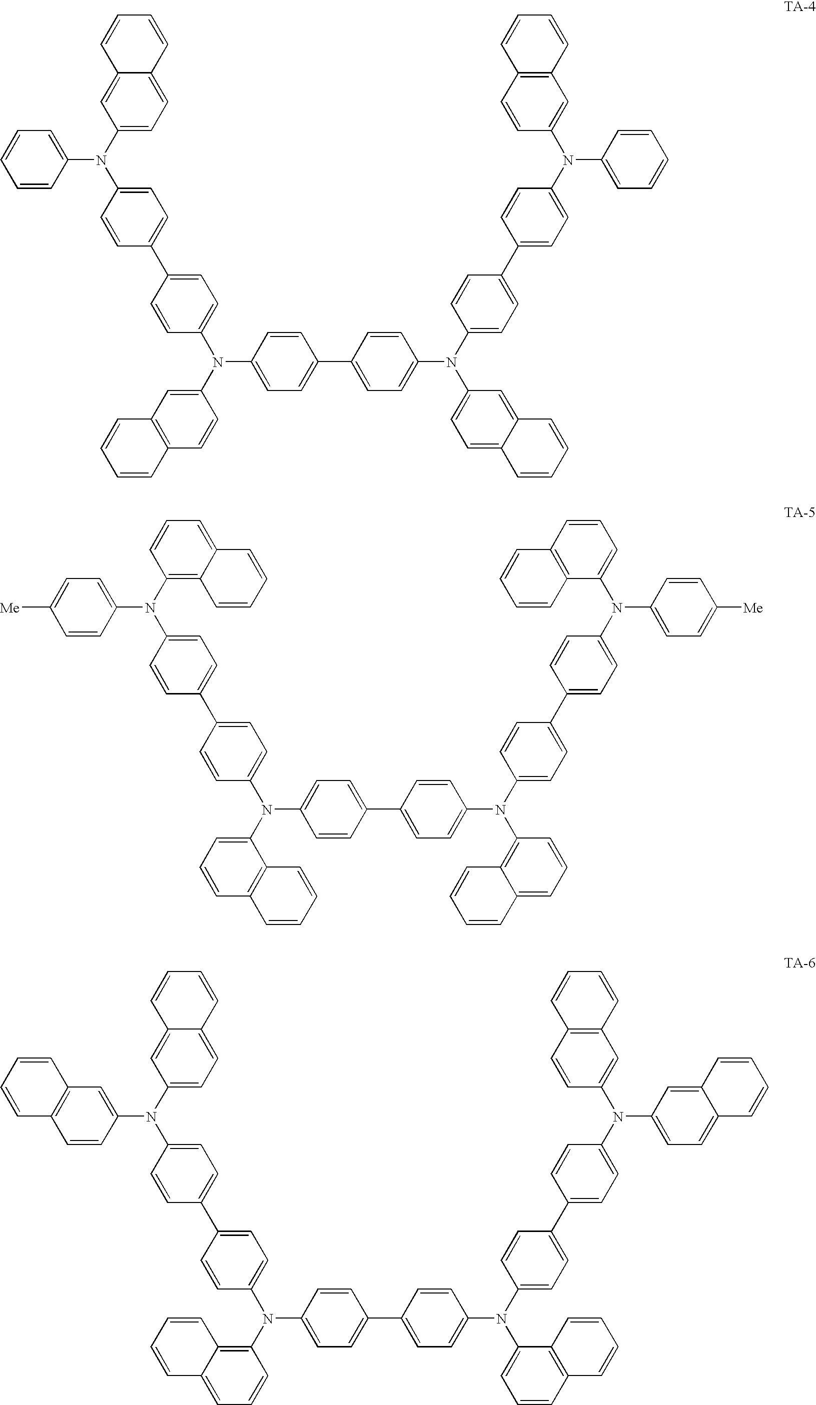

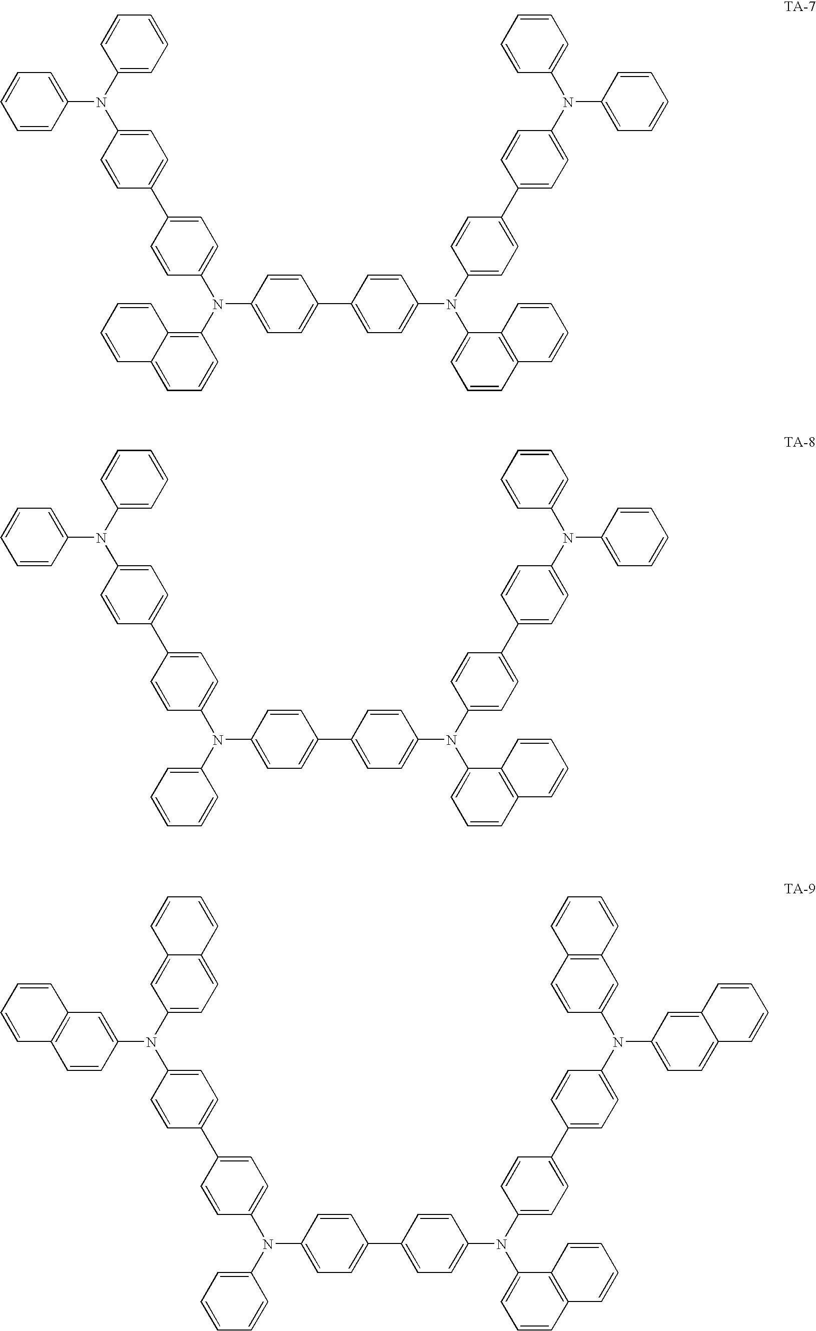

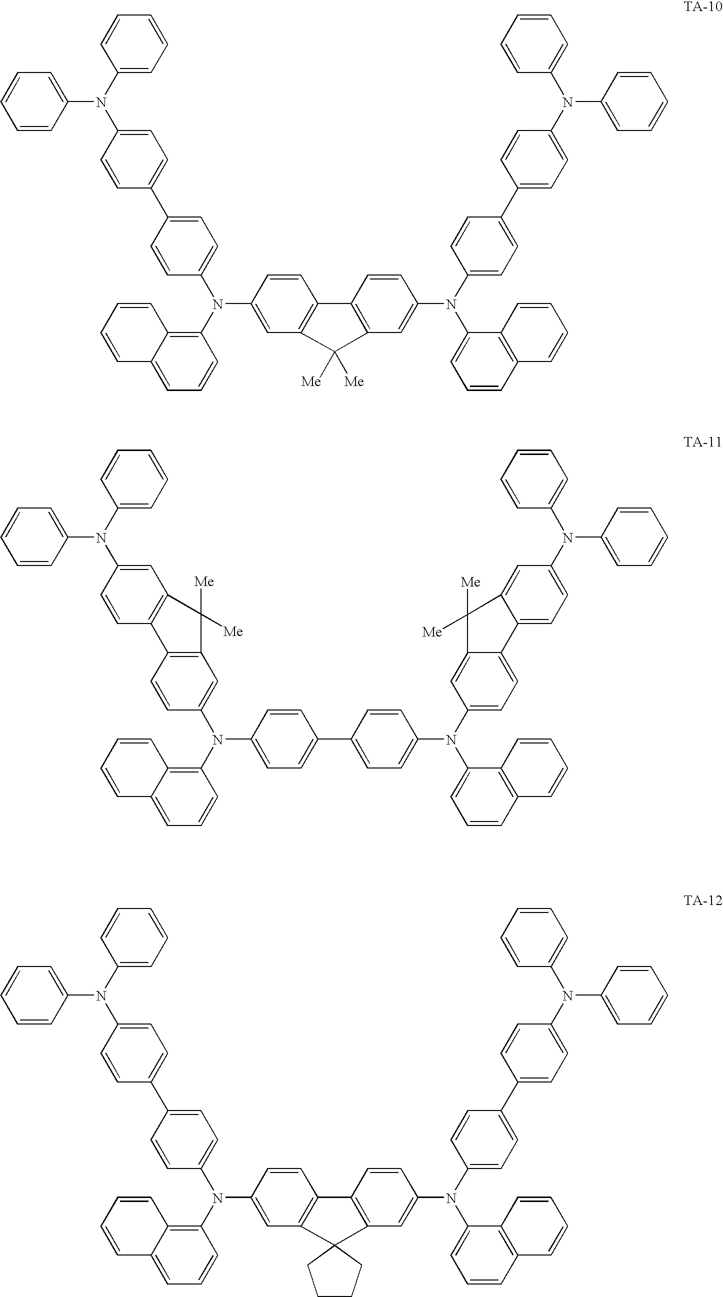

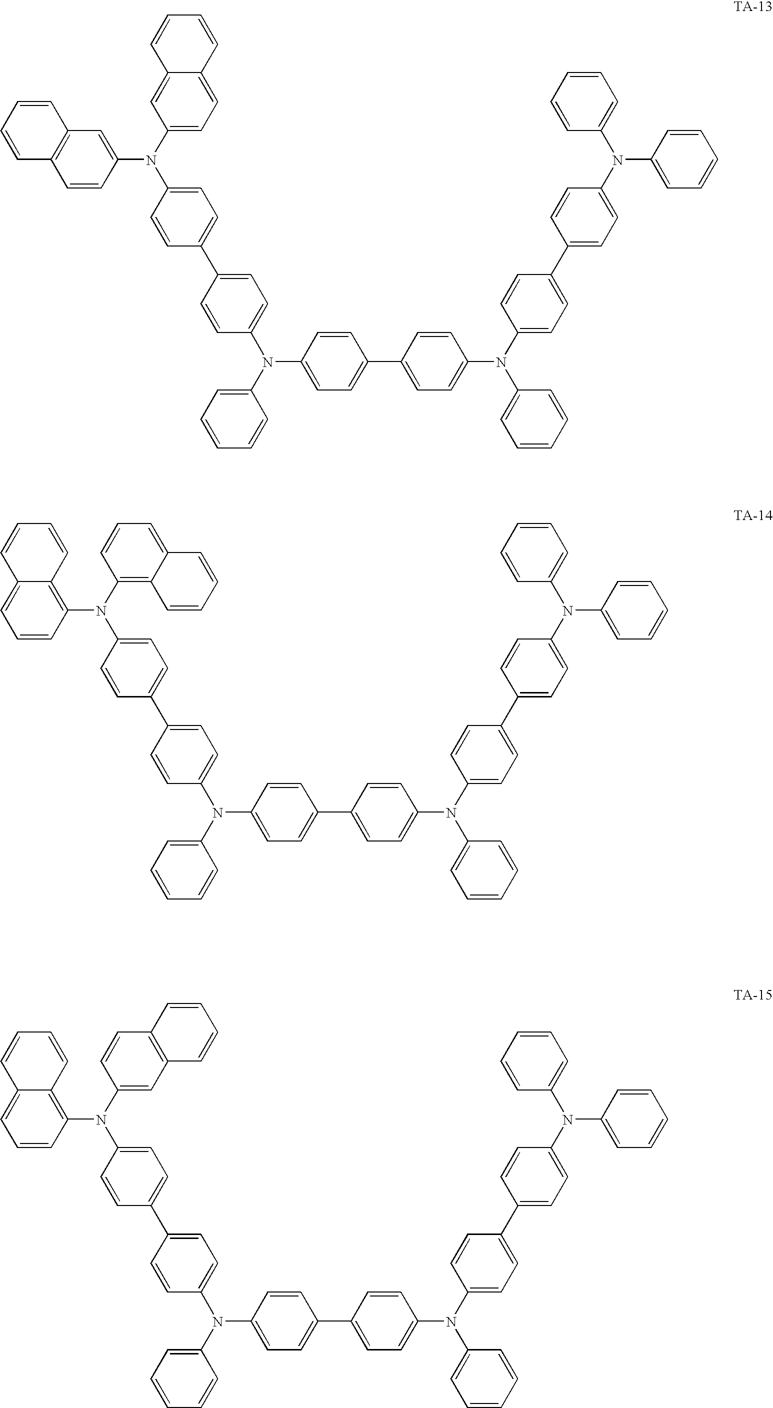

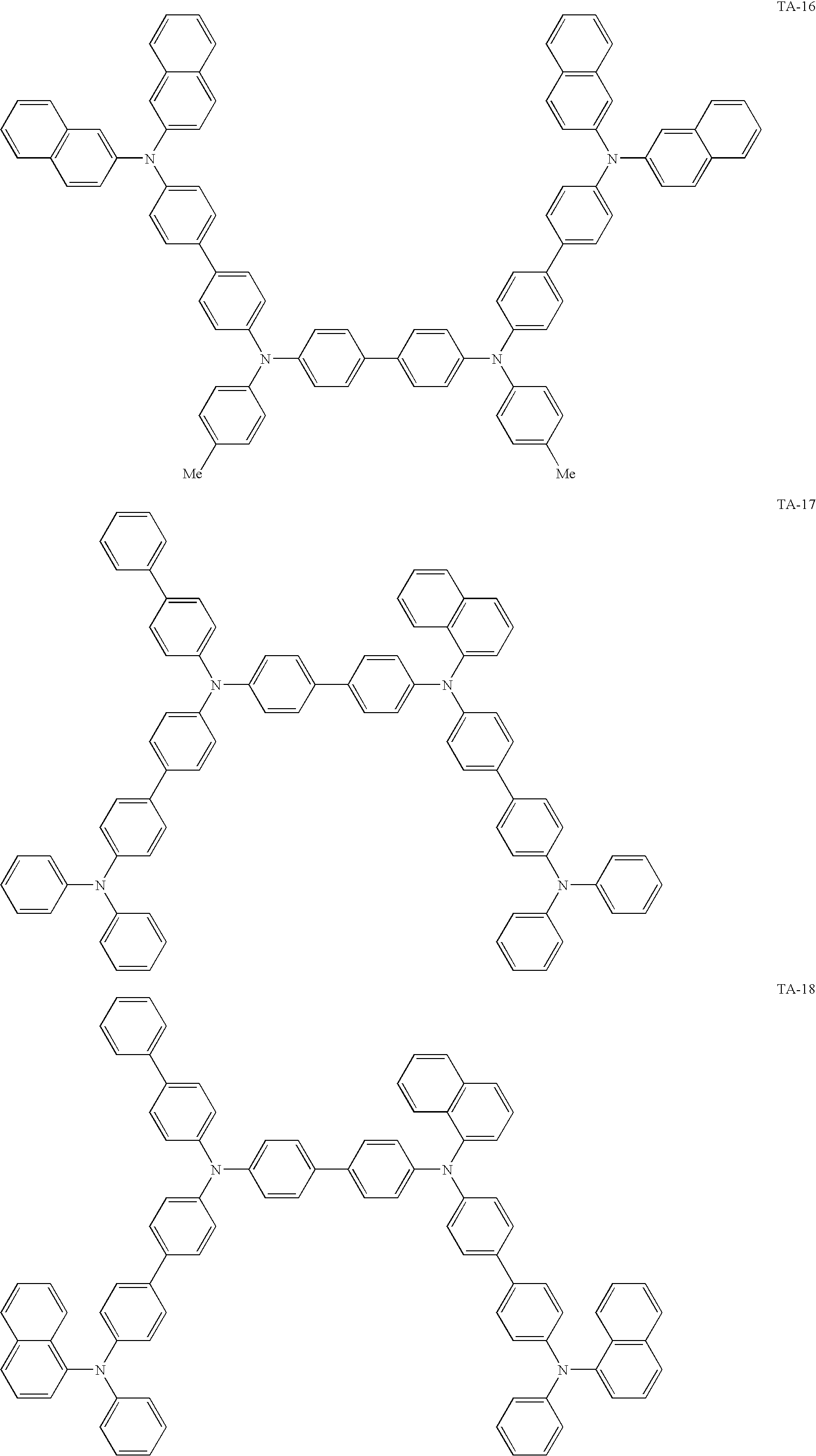

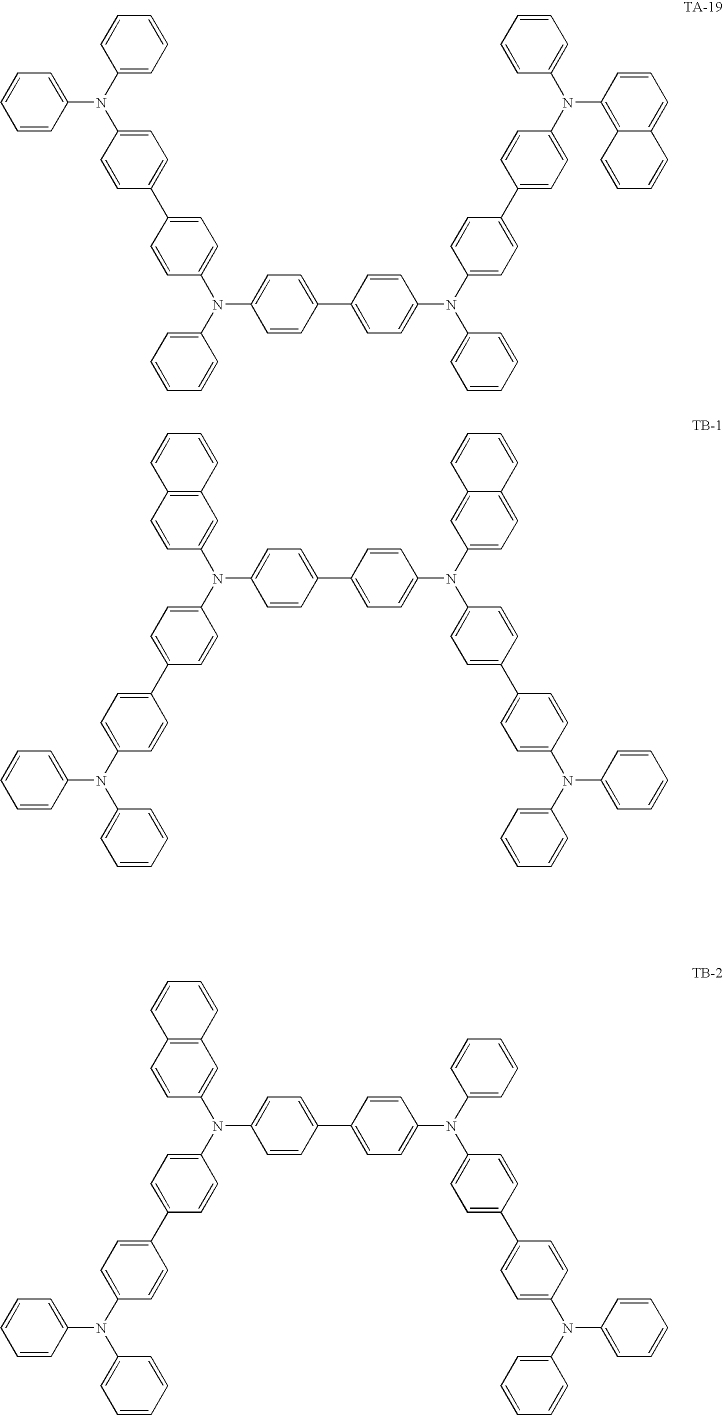

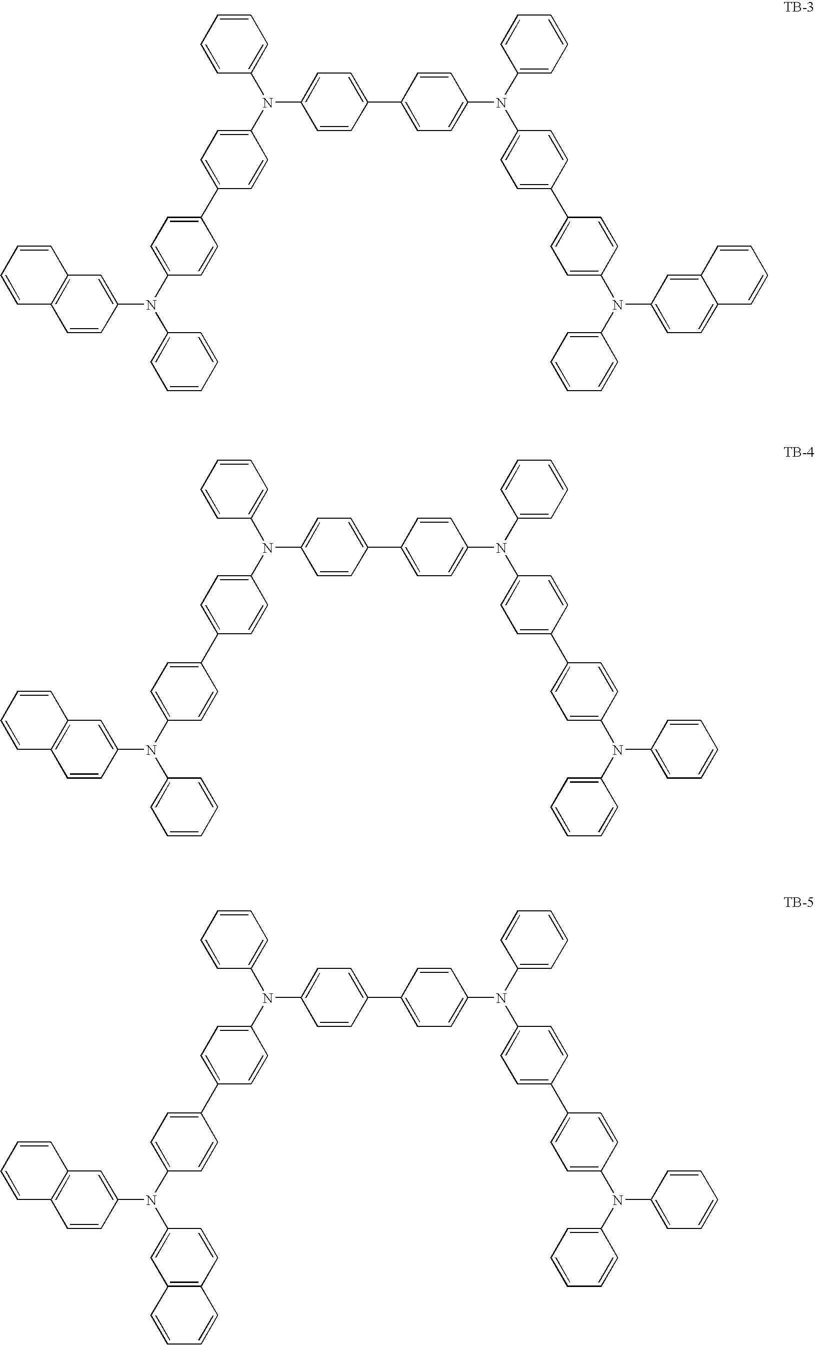

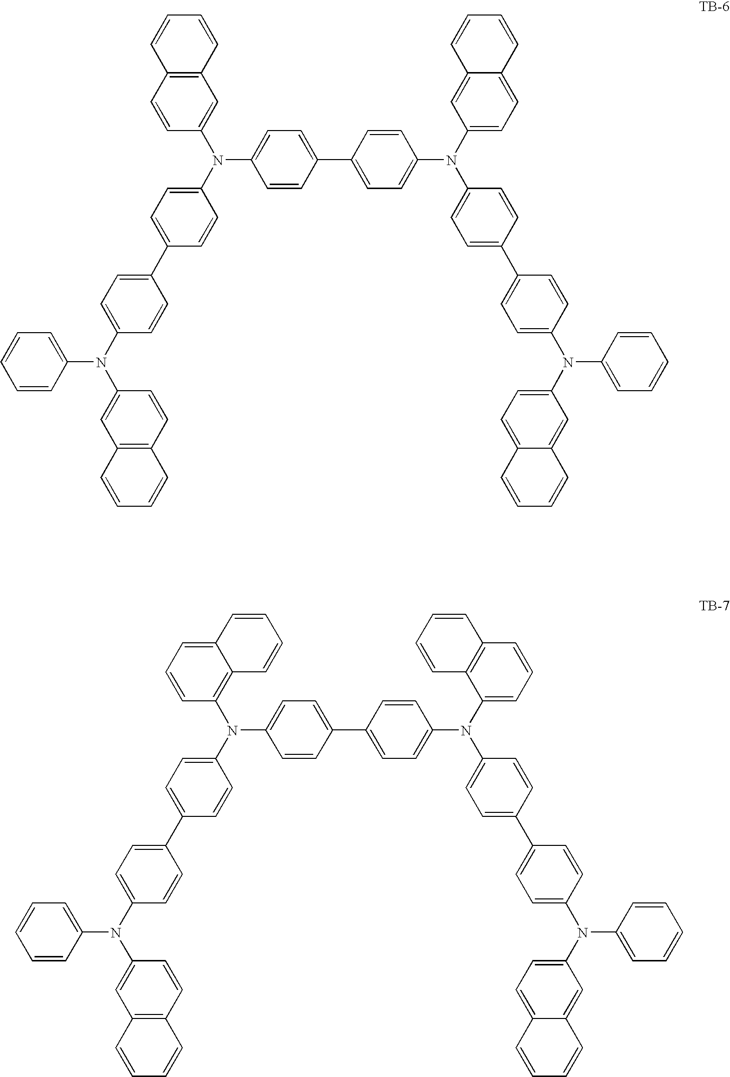

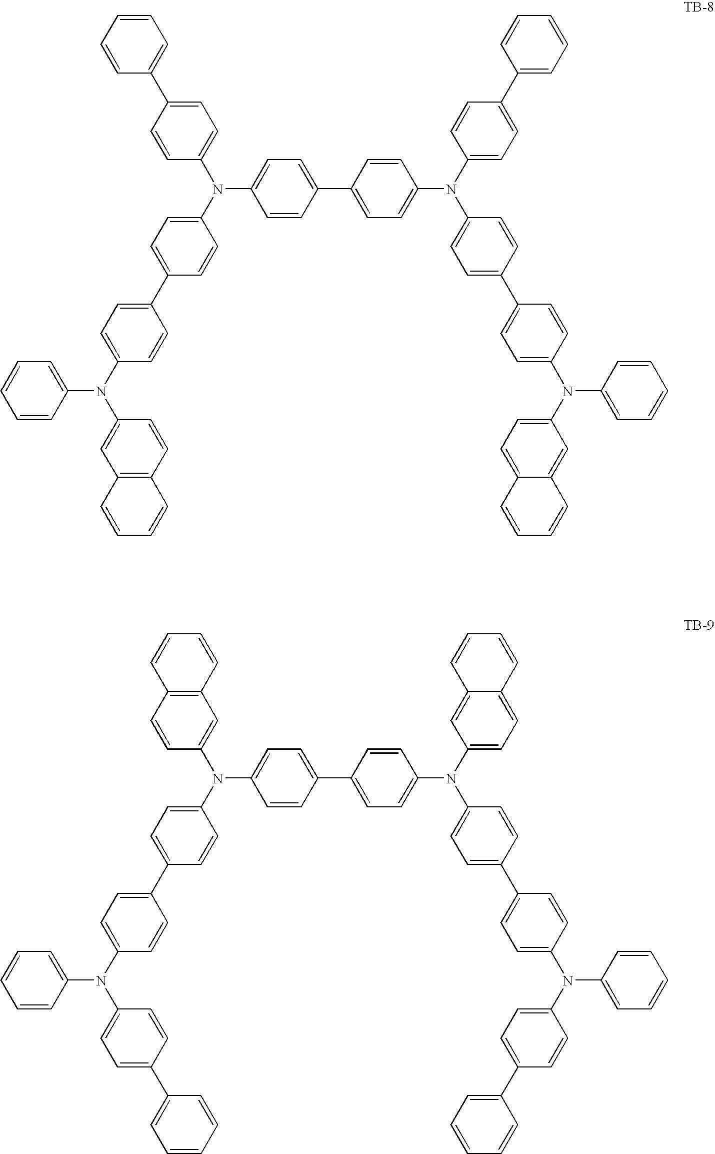

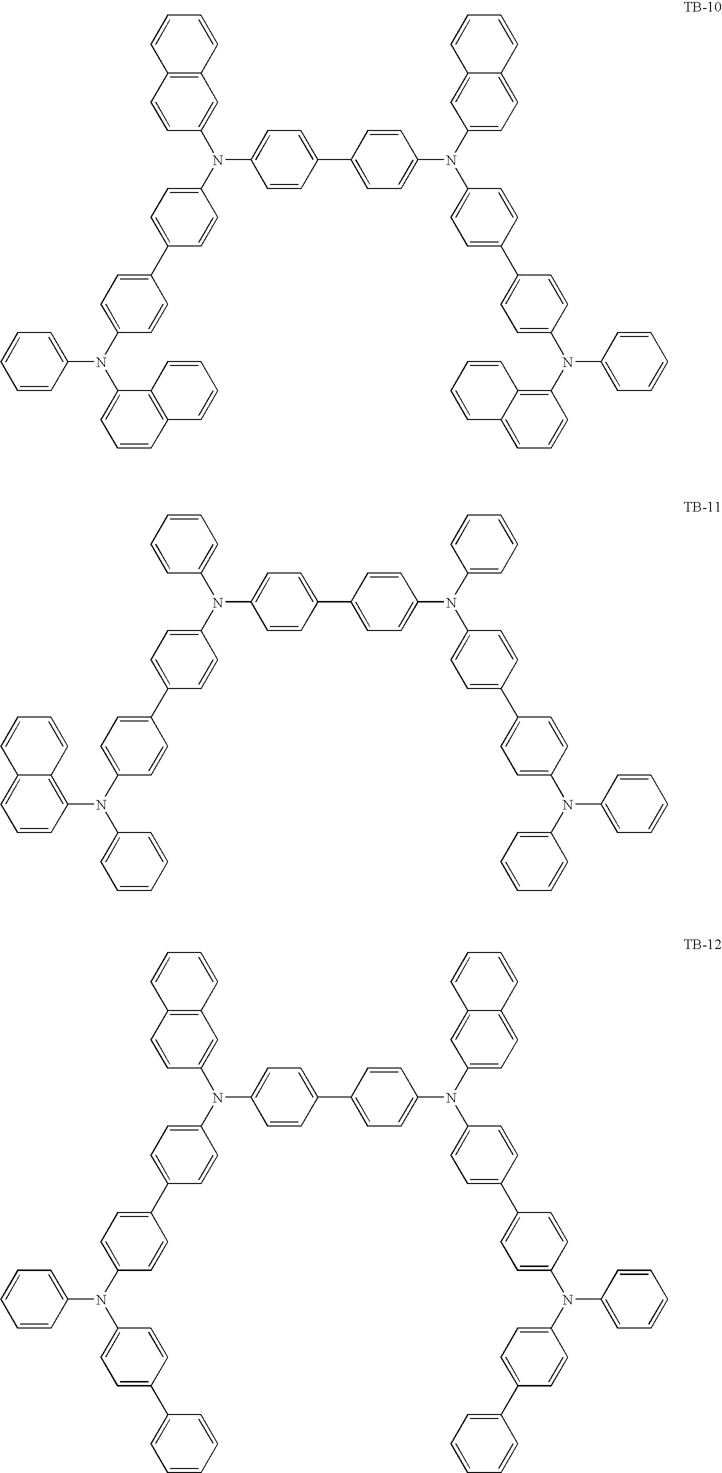

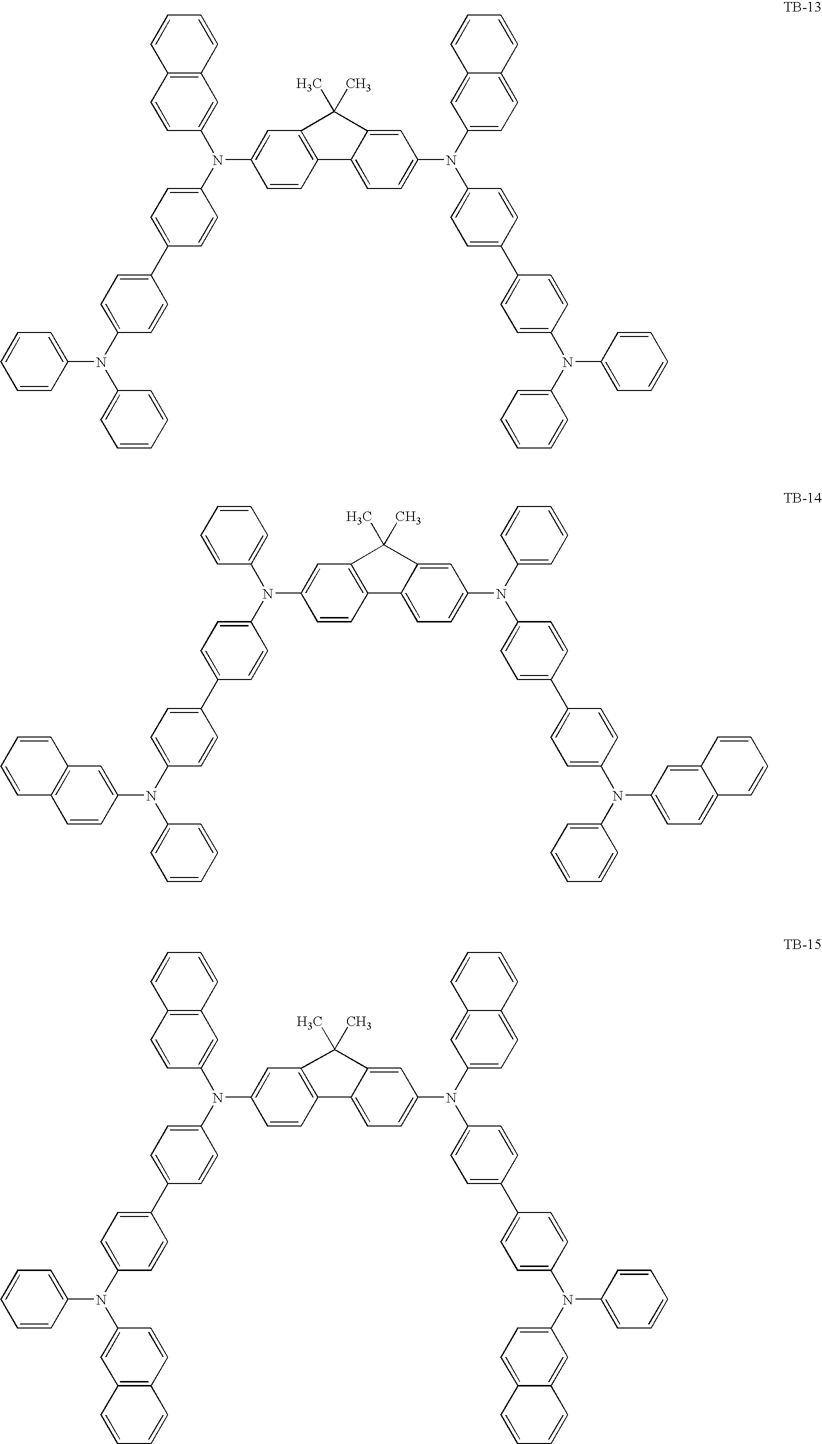

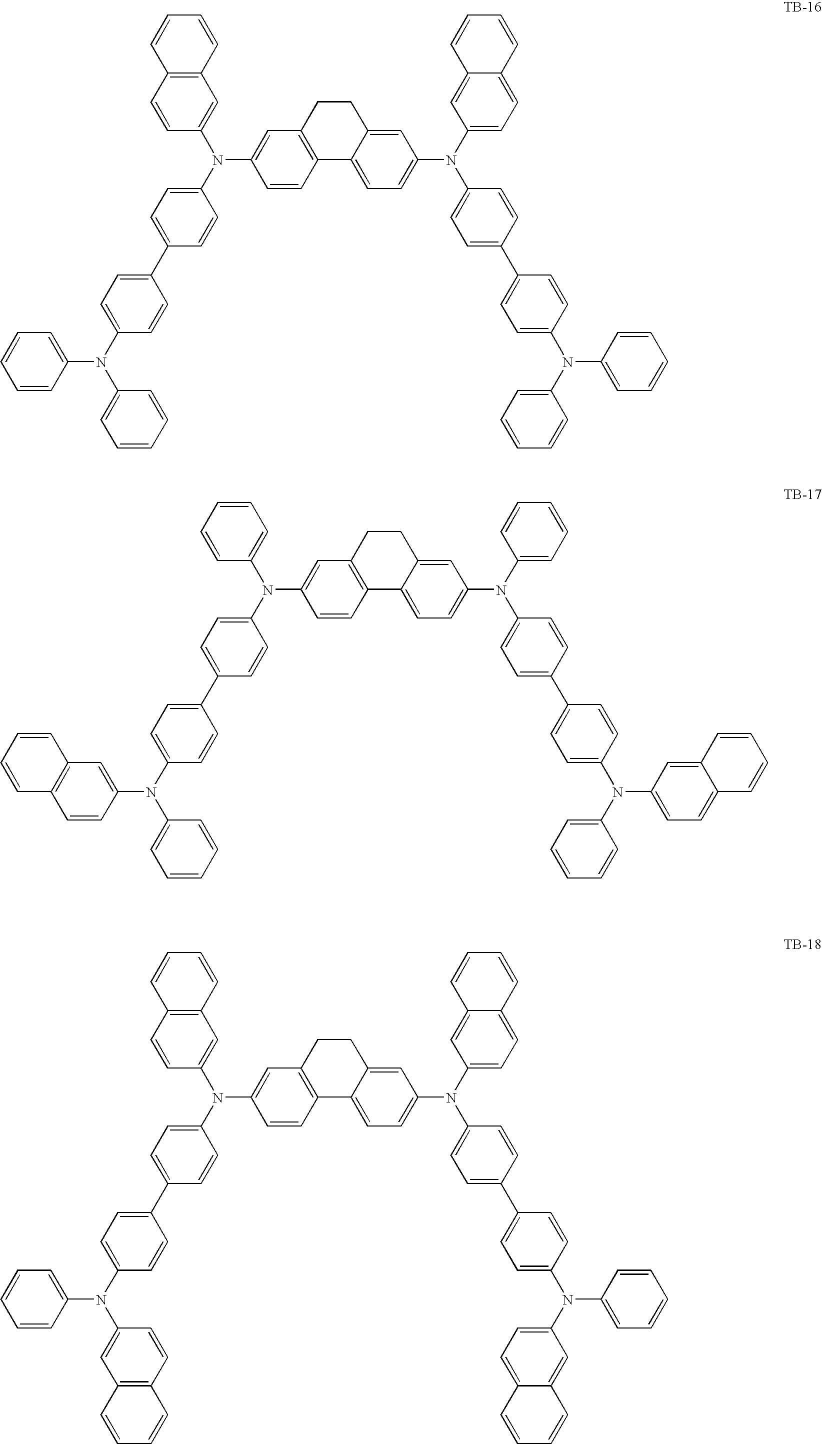

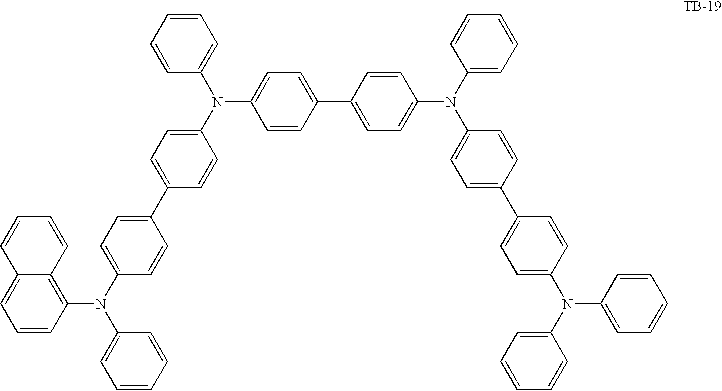

In a third invention of the present invention, at least one layer of the one or multiple organic thin film layers of the organic EL device can be caused to contain the aromatic amine derivative of the present invention alone or as a component of a mixture. The aromatic amine derivative is particularly preferably used in a hole-transporting zone, or is more preferably used in a hole-transporting layer. In such case, an excellent organic EL device can be obtained.

In the organic EL device of the present invention, the layer containing the aromatic amine derivative preferably contacts with the anode.

In the organic EL device of the present invention, the layer contacting with the anode is preferably mainly composed of the aromatic amine derivative.

In the organic EL device of the present invention, the one or multiple organic thin film layers preferably have a layer containing the aromatic amine derivative and a light-emitting material.

In the organic EL device of the present invention, the one or multiple organic thin film layers preferably have a laminate of a hole-transporting layer and/or a hole-injecting layer containing the aromatic amine derivative and a light-emitting layer composed of a phosphorescent metal complex and a host material.

The organic EL device of the present invention preferably emits blue-based light.

The organic EL device of the present invention will be described in detail in the following.

(1) Organic EL Device Constitution

Typical examples of the constitution of the organic EL device of the present invention include the following. Of course, the constitution of the organic EL device of the present invention is not limited to those shown below as the examples.

(1) An anode/light-emitting layer/cathode;

(2) An anode/hole-injecting layer/light-emitting layer/cathode;

(3) An anode/light-emitting layer/electron-injecting layer/cathode;

(4) An anode/hole-injecting layer/light-emitting layer/electron-injecting layer/cathode;

(5) An anode/organic semiconductor layer/light-emitting layer/cathode;

(6) An anode/organic semiconductor layer/electron barrier layer/light-emitting layer/cathode;

(7) An anode/organic semiconductor layer/light-emitting layer/adhesion improving layer/cathode;

(8) An anode/hole-injecting layer/hole-transporting layer/light-emitting layer/electron-injecting layer/cathode;

(9) An anode/insulating layer/light-emitting layer/insulating layer/cathode;

(10) An anode/inorganic semiconductor layer/insulating layer/light-emitting layer/insulating layer/cathode;

(11) An anode/organic semiconductor layer/insulating layer/light-emitting layer/insulating layer/cathode;

(12) An anode/insulating layer/hole-injecting layer/hole-transporting layer/light-emitting layer/insulating layer/cathode; and

(13) An anode/insulating layer/hole-injecting layer/hole-transporting layer/light-emitting layer/electron-injecting layer/a cathode.

Of the above constitutions, the constitution (8) is preferable.

The compound of the present invention may be used in any one of the above organic layers; provided that the compound is preferably incorporated into a light-emitting zone or a hole-transporting zone in those components. The compound is particularly preferably incorporated into a hole-transporting layer. The content of the compound is selected from 30 to 100 mol %.

(2) Transparent Substrate

The organic EL device of the present invention is prepared on a transparent substrate. The transparent substrate is the substrate which supports the organic EL device. It is preferable that the transparent substrate has a transmittance of light of 50% or greater in the visible region of 400 to 700 nm and is flat and smooth.

Examples of the transparent substrate include glass plates and polymer plates. Specific examples of the glass plate include plates made of soda-lime glass, glass containing barium and strontium, lead glass, aluminosilicate glass, borosilicate glass, barium borosilicate glass and quartz. Specific examples of the polymer plate include plates made of polycarbonate resins, acrylic resins, polyethylene terephthalate resins, polyether sulfide resins and polysulfone resins.

(3) Anode

The anode in the organic EL device of the present invention has the function of injecting holes into the hole-transporting layer or the light-emitting layer. It is effective that the anode has a work function of 4.5 eV or greater. Examples of the material for the anode used in the present invention include indium tin oxide alloys (ITO), indium zinc oxide alloys (IZO), tin oxide (NESA), gold, silver, platinum, copper and lanthanoid. Further, alloys thereof and laminates thereof may be used.

The anode can be prepared by forming a thin film of the electrode material described above in accordance with a process such as the vapor deposition process and the sputtering process.

When the light emitted from the light-emitting layer is obtained through the anode, it is preferable that the anode has a transmittance of the emitted light greater than 10%. It is also preferable that the sheet resistivity of the anode is several hundred Ω/□ or smaller. The thickness of the anode is, in general, selected in the range of 10 nm to 1 μm and preferably in the range of 10 to 200 nm although the preferable range may be different depending on the used material.

(4) Light-Emitting Layer

The light-emitting layer in the organic EL device has a combination of the following functions:

(1) The injecting function: the function of injecting holes from the anode or the hole-injecting layer and injecting electrons from the cathode or the electron-injecting layer when an electric field is applied;

(2) The transporting function: the function of transporting injected charges (electrons and holes) by the force of the electric field; and

(3) The light-emitting function: the function of providing the field for recombination of electrons and holes and leading the emission of light. However, the easiness of injection may be different between holes and electrons and the ability of transportation expressed by the mobility may be different between holes and electrons. It is preferable that either one of the charges is transferred.

As the process for forming the light-emitting layer, a conventional process such as the vapor deposition process, the spin coating process and the LB process can be used. It is particularly preferable that the light-emitting layer is a molecular deposit film.

The molecular deposit film is a thin film formed by deposition of a material compound in the gas phase or a thin film formed by solidification of a material compound in a solution or in the liquid phase. In general, the molecular deposit film can be distinguished from the thin film formed in accordance with the LB process (the molecular accumulation film) based on the differences in the aggregation structure and higher order structures and functional differences caused by these structural differences.

Further, as disclosed in JP 57-51781 A, the light-emitting layer can also be formed by dissolving a binder such as a resin and the material compounds into a solvent to prepare a solution, followed by forming a thin film from the prepared solution in accordance with the spin coating process or the like.

In the present invention, where desired, the light-emitting layer may comprise other conventional light-emitting materials other than the light-emitting material comprising the aromatic amine derivative of the present invention, or a light-emitting layer comprising other conventional light-emitting material may be laminated to the light-emitting layer comprising the light-emitting material comprising the aromatic amine derivative of the present invention as long as the object of the present invention is not adversely affected.

Preferable examples of known light-emitting materials include those having a fused aromatic ring such as anthracene and pyrene in the molecule. As shown below, specific examples include anthracene derivatives, asymmetric monoanthracene derivatives, asymmetric anthracene derivatives, and asymmetric pyrene derivatives.

An anthracene derivative as a known light-emitting material is as described below. 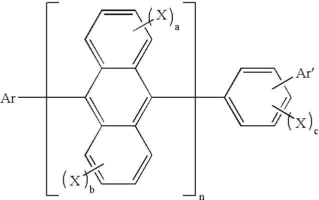
(In the formula, Ar represents a substituted or unsubstituted fused aromatic group having 10 to 50 nuclear carbon atoms. Ar′ represents a substituted or unsubstituted aromatic group having 6 to 50 nuclear carbon atoms. X represents a substituted or unsubstituted aromatic group having 6 to 50 nuclear carbon atoms, a substituted or unsubstituted aromatic heterocyclic group having 5 to 50 nuclear atoms, a substituted or unsubstituted alkyl group having 1 to 50 carbon atoms, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 nuclear atoms, a substituted or unsubstituted arylthio group having 5 to 50 nuclear atoms, a substituted or unsubstituted alkoxycarbonyl group having 1 to 50 carbon atoms, a carboxyl group, a halogen atom, a cyano group, a nitro group, or a hydroxyl group. a, b, and c each represent an integer of 0 to 4. n represents an integer of 1 to 3. In addition, when n represents 2 or more, anthracene nuclei in [] may be identical to or different from each other.)

An asymmetric monoanthracene derivative as a known light-emitting material has the following structure. 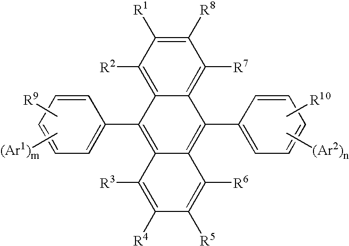
(In the formula, Ar¹ and Ar² each independently represent a substituted or unsubstituted aromatic ring group having 6 to 50 nuclear carbon atoms. m and n each represent an integer of 1 to 4. Ar¹ and Ar² are not identical to each other when m=n=1 and positions at which Ar¹ and Ar² are bound to a benzene ring are bilaterally symmetric, and m and n represent different integers when m or n represents an integer of 2 to 4. R¹ to R¹⁰ each independently represent a hydrogen atom, a substituted or unsubstituted aromatic ring group having 6 to 50 nuclear carbon atoms, a substituted or unsubstituted aromatic heterocyclic group having 5 to 50 nuclear atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 nuclear atoms, a substituted or unsubstituted arylthio group having 5 to 50 nuclear atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group.)

An asymmetric anthracene derivative as a known light-emitting material has the following structure. 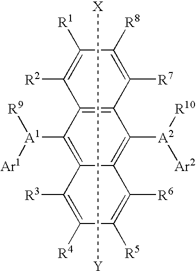
(In the formula, A¹ and A² each independently represent a substituted or unsubstituted fused aromatic ring group having 10 to 20 nuclear carbon atoms. Ar¹ and Ar² each independently represent a hydrogen atom, or a substituted or unsubstituted aromatic ring group having 6 to 50 nuclear carbon atoms. R¹ to R¹⁰ each independently represent a hydrogen atom, a substituted or unsubstituted aromatic ring group having 6 to 50 nuclear carbon atoms, a substituted or unsubstituted aromatic heterocyclic group having 5 to 50 nuclear atoms, a substituted or unsubstituted alkyl group having 1 to 50 carbon atoms, a substituted or unsubstituted cycloalkyl group, a substituted or unsubstituted alkoxy group having 1 to 50 carbon atoms, a substituted or unsubstituted aralkyl group having 6 to 50 carbon atoms, a substituted or unsubstituted aryloxy group having 5 to 50 nuclear atoms, a substituted or unsubstituted arylthio group having 5 to 50 nuclear atoms, a substituted or unsubstituted alkoxycarbonyl group having 1 to 50 carbon atoms, a substituted or unsubstituted silyl group, a carboxyl group, a halogen atom, a cyano group, a nitro group, or a hydroxyl group. The number of each of Ar¹, Ar² R⁹, and R¹⁰ maybe two or more, and adjacent groups may form a saturated or unsaturated cyclic structure; provided that the case where groups symmetric with respect to the X-Y axis shown on central anthracene in the general formula (I) bind to 9- and 10-positions of the anthracene does not occur.)

An asymmetric pyrene derivative as a known light-emitting material has the following structure. 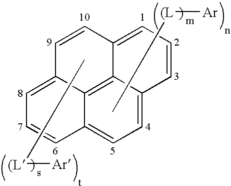
[In the formula, Ar and Ar′ each represent a substituted or unsubstituted aromatic group having 6 to 50 nuclear carbon atoms. L and L′ each represent a substituted or unsubstituted phenylene group, a substituted or unsubstituted naphthalenylene group, a substituted or unsubstituted fluorenylene group, or a substituted or unsubstituted dibenzosilolylene group. m represents an integer of 0 to 2. n represents an integer of 1 to 4. s represents an integer of 0 to 2. t represents an integer of 0 to 4. In addition, L or Ar binds to any one of 1- to 5-positions of pyrene, and L′ or Ar′ binds to any one of 6- to 10-positions of pyrene; provided that Ar, Ar′, L, and L′ satisfy the following item (1) or (2) when n+t represents an even number.

(1) Ar≠Ar′ and/or L≠L′ (where the symbol “≠” means that groups connected with the symbol have different structures)

(2) When Ar═Ar′ and L=L′,

(2-1) m≠s and /or n≠t, or

(2-2) when m=s and n=t,

(2-2-1) L and L′, or pyrene, bind to different binding positions on Ar and Ar′, or

(2-2-2) in the case where L and L′, or pyrene, bind to the same binding positions on Ar and Ar′, the case where the substitution positions of L and L′, or of Ar and Ar′ in pyrene are 1- and 6-positions, or 2- and 7-positions does not occur.]

(5) Hole-Injecting and Transporting Layer

The hole-injecting and transporting layer is a layer which helps injection of holes into the light-emitting layer and transports the holes to the light-emitting region. The layer exhibits a great mobility of holes and, in general, has an ionization energy as small as 5.5 eV or smaller. For the hole-injecting and transporting layer, a material which transports holes to the light-emitting layer under an electric field of a smaller strength is preferable. A material which exhibits, for example, a mobility of holes of at least 10⁻⁴ cm²/V·sec under application of an electric field of 10⁴ to 10⁶ V/cm is preferable.

When the aromatic amine derivative of the present invention is used in the hole-transporting zone, the aromatic amine derivative of the present invention may be used alone or as a mixture with other materials for forming the hole-transporting and injecting layer.

The other material which can be used for forming the hole-transporting and injecting layer as a mixture with the aromatic amine derivative of the present invention is not particularly limited as long as the material has a described property. The other material can be selected as desired from materials which are conventionally used as the charge transporting material of holes in photoconductive materials and conventional materials which are used for the hole-injecting layer in organic EL devices.

Specific examples include: a triazole derivative (see, for example U.S. Pat. No. 3,112,197); an oxadiazole derivative (see, for example U.S. Pat. No. 3,189,447); an imidazole derivative (see, for example JP-B-37-16096); a polyarylalkane derivative (see, for example U.S. Pat. No. 3,615,402, U.S. Pat. No. 3,820,989 and U.S. Pat. No. 3,542,544, JP-B-45-555, JP-B-51-10983, JP-A-51-93224, JP-A-55-17105, JP-A-56-4148, JP-A-55-108667, JP-A-55-156953, and JP-A-56-36656); a pyrazoline derivative and a pyrazolone derivative (see, for example U.S. Pat. No. 3,180,729, U.S. Pat. No. 4,278,746, JP-A-55-88064, JP-A-55-88065, JP-A-49-105537, JP-A-55-51086, JP-A-56-80051, JP-A-56-88141, JP-A-57-45545, JP-A-54-112637, and JP-A-55-74546); a phenylenediamine derivative (see, for example U.S. Pat. No. 3,615,404, JP-B-51-10105, JP-B-46-3712, JP-B-47-25336, JP-A-54-53435, JP-A-54-110536, and JP-A-54-119925); an arylamine derivative (see, for example U.S. Pat. No. 3,567,450, U.S. Pat. No. 3,180,703, U.S. Pat. No. 3,240,597, U.S. Pat. No. 3,658,520, U.S. Pat. No. 4,232,103, U.S. Pat. No. 4,175,961, U.S. Pat. No. 4,012,376, JP-B-49-35702, JP-B-39-27577, JP-A-55-144250, JP-A-56-119132, JP-A-56-22437, and DE 1,110,518); an amino-substituted chalcone derivative (see, for example U.S. Pat. No. 3,526,501); an oxazole derivative (those disclosed in U.S. Pat. No. 3,257,203); a styrylanthracene derivative (see, for example JP-A-56-46234); a fluorenone derivative (see, for example JP-A-54-110837); a hydrazone derivative (see, for example U.S. Pat. No. 3,717,462, JP-A-54-59143, JP-A-55-52063, JP-A-55-52064, JP-A-55-46760, JP-A-55-85495, JP-A-57-11350, JP-A-57-148749, and JP-A-2-311591); a stilbene derivative (see, for example JP-A-61-210363, JP-A-61-228451, JP-A-61-14642, JP-A-61-72255, JP-A-62-47646, JP-A-62-36674, JP-A-62-10652, JP-A-62-30255, JP-A-60-93445, JP-A-60-94462, JP-A-60-174749, and JP-A-60-175052); a silazane derivative (U.S. Pat. No. 4,950,950); a polysilane-based (JP-A-2-204996); an aniline-based copolymer (JP-A-2-282263); and an electroconductive high molecular weight oligomer (particularly a thiophene oligomer) disclosed in JP-A-1-211399.

In addition to the above materials which can be used as the material for the hole-injecting layer, a porphyrin compound (those disclosed in, for example, JP-A-63-295695); an aromatic tertiary amine compound and a styrylamine compound (see, for example U.S. Pat. No. 4,127,412, JP-A-53-27033, JP-A-54-58445, JP-A-54-149634, JP-A-54-64299, JP-A-55-79450, JP-A-55-144250, JP-A-56-119132, JP-A-61-295558, JP-A-61-98353, and JP-A-63-295695); are preferable, and aromatic tertiary amines are particularly preferable.

Further examples of aromatic tertiary amine compounds include compounds having two fused aromatic rings in the molecule such as 4,4′-bis(N-(1-naphthyl)-N-phenylamino)-biphenyl (hereinafter referred to as NPD) as disclosed in U.S. Pat. No. 5,061,569, and a compound in which three triphenylamine units are bonded together in a star-burst shape, such as 4,4′,4″-tris(N-(3-methylphenyl)-N-phenylamino)-triphenylamine (hereinafter referred to as MTDATA) as disclosed in JP-A-4-308688 can be exemplified.

Further, the aromatic dimethylidene-based compounds described above as the examples of the material for the light-emitting layer and inorganic compounds such as Si of the p-type and SiC of the p-type can also be used as the material for the hole-injecting layer.

The hole-injecting and transporting layer can be formed by the compound described above in accordance with a conventional process such as the vacuum vapor deposition process, the spin coating process, the casting process and the LB process. The thickness of the hole-injecting and transporting layer is not particularly limited. In general, the thickness is 5 nm to 5 μm. The hole-injecting and transporting layer may comprise a single layer comprising one or more materials described above or may be a laminate comprising a hole-injecting and transporting layer comprising materials different from the materials of the hole-injecting and transporting layer described above as long as the compound of the present invention is comprised in the hole-injecting and transporting zone.

Further, an organic semiconductor layer may be disposed as a layer helping the injection of holes or electrons into the light-emitting layer. As the organic semiconductor layer, a layer having a conductivity of 10⁻¹⁰ S/cm or greater is preferable. As the material for the organic semiconductor layer, oligomers containing thiophene, and conductive oligomers such as arylamine oligomers and conductive dendrimers such as arylamine dendrimers, which are disclosed in JP-A-08-193191, can be used.

(6) Electron-Injecting Layer

The electron-injecting layer is a layer which helps injection of electrons into the light-emitting layer and exhibits a great mobility of electrons. The adhesion improving layer is an electron-injecting layer especially comprising a material exhibiting improved adhesion with the cathode. As the material used for the electron-injecting layer, metal complexes of 8-hydroxyquinoline and derivatives thereof are preferable.

Examples of the metal complex of 8-hydroxyquinoline or the derivatives described above include metal chelate oxinoide compounds containing the chelate of oxine (in general, 8-quinolinol or 8-hydroxyquinoline).

For example, Alq described in the light-emitting material section may be used as an electron-injecting layer.

On the other hand, examples of the oxadiazole derivative include electron transfer compounds represented by the following general formulae: 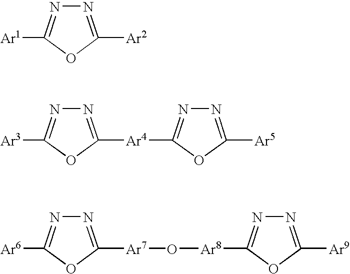
(In the formulae, Ar¹, Ar², Ar³, Ar⁵, Ar⁶ and Ar⁹ each represent a substituted or unsubstituted aryl group and may represent the same group or different groups. Ar⁴, Ar⁷ and Ar⁸ each represent a substituted or unsubstituted arylene group and may represent the same group or different groups)

Examples of the aryl group include phenyl group, biphenyl group, anthryl group, perylenyl group and pyrenyl group. Examples of the arylene group include phenylene group, naphthylene group, biphenylene group, anthrylene group, perylenylene group and pyrenylene group. Examples of the substituent include alkyl groups having 1 to 10 carbon atoms, alkoxyl groups having 1 to 10 carbon atoms and cyano group. As the electron transfer compound, compounds which can form thin films are preferable. Examples of the electron transfer compounds described above include the following.

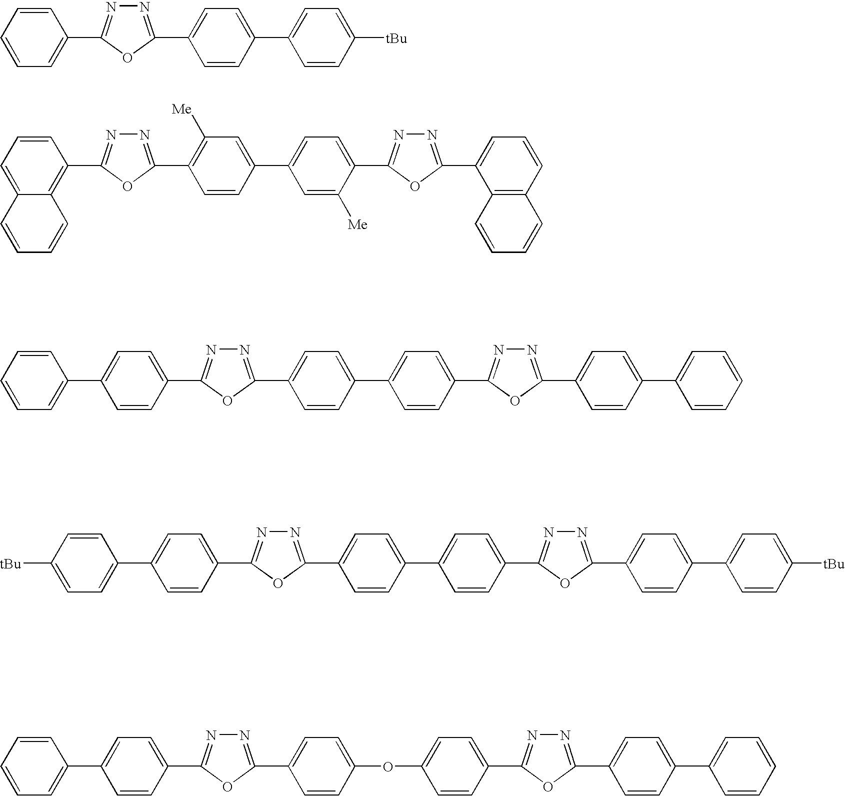

Further, other compounds having a nitrogen-containing heterocyclic ring to be preferable as an electron-transporting material are known. Known examples of such a nitrogen-containing heterocyclic derivative include a nitrogen-containing heterocyclic derivative described below.

Preferable examples of the electron transfer material include nitrogen-containing heterocyclic ring derivatives each represented by the following formula.
HAr-L-Ar¹-Ar²

(In the formula, HAr represents a nitrogen-containing heterocyclic ring having 3 to 40 carbon atoms which may have a substituent, L represents a single bond, an arylene group having 6 to 60 carbon atoms which may have a substituent, a heteroarylene group having 3 to 60 carbon atoms which may have a substituent, or a fluorenylene group which may have a substituent, Ar¹ represents a divalent aromatic hydrocarbon group having 6 to 60 carbon atoms which may have a substituent, and Ar2represents an aryl group having 6 to 60 carbon atoms which may have a substituent, or a heteroaryl group having 3 to 6.0 carbon atoms which may have a substituent.)

Further, an electron transfer material including a nitrogen-containing heterocyclic ring such as that shown in either of the following two formulae constructions is also favorable. 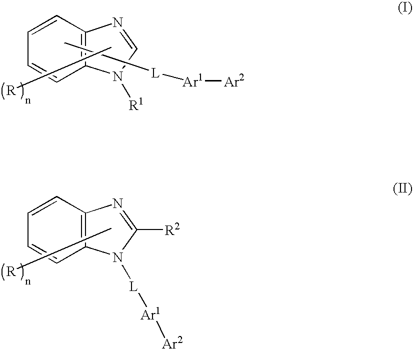
(In the formula, Rs each represent a hydrogen atom, an aryl group having 6 to 60 carbon atoms which may have a substituent, a pyridyl group which may have a substituent, a quinolyl group which may have a substituent, an alkyl group having 1 to 20 carbon atoms which may have a substituent, or an alkoxy group having 1 to 20 carbon atoms which may have a substituent. n represents an integer of 0 to 4. R¹ represents an aryl group having 6 to 60 carbon atoms which may have a substituent, a pyridyl group which may have a substituent, a quinolyl group which may have a substituent, an alkyl group having 1 to 20 carbon atoms which may have a substituent, or an alkoxy group having 1 to 20 carbon atoms. R² represents a hydrogen atom, an aryl group having 6 to 60 carbon atoms which may have a substituent, a pyridyl group which may have a substituent, a quinolyl group which may have a substituent, an alkyl group having 1 to 20 carbon atoms which may have a substituent, or an alkoxy group having 1 to 20 carbon atoms which may have a substituent. L represents an arylene group having 6 to 60 carbon atoms which may have a substituent, a pyridinylene group which may have a substituent, a quinolinylene group which may have a substituent, or a fluorenylene group which may have a substituent. Ar¹ represents an arylene group having 6 to 60 carbon atoms which may have a substituent, a pyridinylene group which may have a substituent, or a quinolinylene group which may have a substituent. Ar² represents an aryl group having 6 to 60 carbon atoms which may have a substituent, a pyridyl group which may have a substituent, a quinolyl group which may have a substituent, an alkyl group having 1 to 20 carbon atoms which may have a substituent, or an alkoxy group having 1 to 20 carbon atoms which may have a substituent.)

A preferable embodiment of the present invention includes an element comprising a reducing dopant in the region of electron transport or in the interfacial region of the cathode and the organic thin film layer. The reducing dopant is defined as a substance which can reduce a compound having the electron-transporting property. Various compounds can be used as the reducing dopant as long as the compounds have a uniform reductive property. For example, at least one substance selected from the group consisting of alkali metals, alkaline earth metals, rare earth metals, alkali metal oxides, alkali metal halides, alkaline earth metal oxides, alkaline earth metal halides, rare earth metal oxides, rare earth metal halides, organic complexes of alkali metals, organic complexes of alkaline earth metals and organic complexes of rare earth metals can be advantageously used.

More specifically, examples of the reducing dopant includes substances having a work function of 2.9 eV or smaller, specific examples of which include at least one alkali metal selected from the group consisting of Na (the work function: 2.36 eV), K (the work function: 2.28 eV), Rb (the work function: 2.16 eV) and Cs (the work function: 1.95 eV) and at least one alkaline earth metal selected from the group consisting of Ca (the work function: 2.9 eV), Sr (the work function: 2.0 to 2.5 eV) and Ba (the work function: 2.52 eV). Among the above substances, at least one alkali metal selected from the group consisting of K, Rb and Cs is more preferable, Rb and Cs are still more preferable, and Cs is most preferable as the reducing dopant. These alkali metals have great reducing ability, and the luminance of the emitted light and the life of the organic EL device can be increased by addition of a relatively small amount of the alkali metal into the electron-injecting zone. As the reducing dopant having a work function of 2.9 eV or smaller, combinations of two or more alkali metals thereof are also preferable. Combinations having Cs such as the combinations of Cs and Na, Cs and K, Cs and Rb and Cs, Na and K are more preferable. The reducing ability can be efficiently exhibited by the combination having Cs. The luminance of emitted light and the life of the organic EL device can be increased by adding the combination having Cs into the electron-injecting zone.

The present invention may further comprise an electron-injecting layer which is constituted with an insulating material or a semiconductor and disposed between the cathode and the organic layer. At this time, leak of electric current can be effectively prevented by the electron-injecting layer and the electron-injecting property can be improved. As the insulating material, at least one metal compound selected from the group consisting of alkali metal chalcogenides, alkaline earth metal chalcogenides, alkali metal halides and alkaline earth metal halides is preferable. It is preferable that the electron-injecting layer is constituted with the above substance such as the alkali metal chalcogenide since the electron-injecting property can be further improved. Preferable examples of the alkali metal chalcogenide include Li₂O, K₂O, Na₂S, Na₂Se and Na₂O. Preferable examples of the alkaline earth metal chalcogenide include CaO, BaO, SrO, BeO, BaS and CaSe. Preferable examples of the alkali metal halide include LiF, NaF, KF, LiCl, KCl and NaCl. Preferable examples of the alkaline earth metal halide include fluoride such as CaF₂, BaF₂, SrF₂, MgF₂ and BeF₂ and halides other than the fluorides.

Examples of the semiconductor constituting the electron-transporting layer include oxides, nitrides and oxide nitrides of at least one element selected from Ba, Ca, Sr, Yb, Al, Ga, In, Li, Na, Cd, Mg, Si, Ta, Sb and Zn used singly or in combination of two or more. It is preferable that the inorganic compound constituting the electron-transporting layer forms a crystallite or amorphous insulating thin film. When the electron-injecting layer is constituted with the insulating thin film described above, a more uniform thin film can be formed, and defects of pixels such as dark spots can be decreased. Examples of the inorganic compound include alkali metal chalcogenides, alkaline earth metal chalcogenides, alkali metal halides and alkaline earth metal halides which are described above.

(7) Cathode

As the cathode, a material such as a metal, an alloy, a conductive compound or a mixture of these materials which has a small work function (4 eV or smaller) is used as the electrode material. Examples of the electrode material include sodium, sodium-potassium alloys, magnesium, lithium, magnesium-silver alloys, aluminum/aluminum oxide, aluminum-lithium alloys, indium and rare earth metals.

The cathode can be prepared by forming a thin film of the electrode material described above in accordance with a process such as the vapor deposition process and the sputtering process.

When the light emitted from the light-emitting layer is obtained through the cathode, it is preferable that the cathode has a transmittance of the emitted light greater than 10%.

It is also preferable that the sheet resistivity of the cathode is several hundred Ω/□ or smaller. The thickness of the cathode is, in general, selected in the range of 10 nm to 1 μm and preferably in the range of 50 to 200 nm.

(8) Insulating Layer

Defects in pixels tend to be formed in organic EL device due to leak and short circuit since an electric field is applied to ultra-thin films. To prevent the formation of the defects, a layer of a thin film having an insulating property may be inserted between the pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, vanadium oxide, and cesium carbonate.

Mixtures and laminates of the above compounds can also be used.

(9) Process for Producing the Organic EL Device

To prepare the organic EL device of the present invention, for example, the anode, the light-emitting layer and, where necessary, the hole-injecting layer and the electron-injecting layer are formed in accordance with the illustrated process using the illustrated materials, and the cathode is formed in the last step. The organic EL device may also be prepared by forming the above layers in the order reverse to that described above, i.e., the cathode being formed in the first step and the anode in the last step.

Hereinafter, an embodiment of the process for preparing an organic EL device having a construction in which an anode, a hole-injecting layer, a light-emitting layer, an electron-injecting layer and a cathode are disposed successively on a transparent substrate will be described.

On a suitable transparent substrate, a thin film made of a material for the anode is formed in accordance with the vapor deposition process or the sputtering process so that the thickness of the formed thin film is 1 μm or smaller and preferably in the range of 10 to 200 nm. The formed thin film is used as the anode. Then, a hole-injecting layer is formed on the anode. The hole-injecting layer can be formed in accordance with the vacuum vapor deposition process, the spin coating process, the casting process or the LB process, as described above. The vacuum vapor deposition process is preferable since a uniform film can be easily obtained and the possibility of formation of pin holes is small. When the hole-injecting layer is formed in accordance with the vacuum vapor deposition process, in general, it is preferable that the conditions are suitably selected in the following ranges: the temperature of the source of the deposition: 50 to 450° C.; the vacuum: 10⁻⁷ to 10⁻³ Torr; the rate of deposition: 0.01 to 50 nm/second; the temperature of the substrate: −50 to 300° C. and the thickness of the film: 5 nm to 5 μm; although the conditions of the vacuum vapor deposition are different depending on the used compound (the material for the hole-injecting layer) and the crystal structure and the recombination structure of the hole-injecting layer to be formed.

Then, the light-emitting layer is formed on the hole-injecting layer formed above. Using a desired organic light-emitting material, a thin film of the organic light-emitting material can be formed in accordance with the vacuum vapor deposition process, the sputtering process, the spin coating process or the casting process, and the formed thin film is used as the light-emitting layer. The vacuum vapor deposition process is preferable since a uniform film can be easily obtained and the possibility of formation of pin holes is small. When the light-emitting layer is formed in accordance with the vacuum vapor deposition process, in general, the conditions of the vacuum vapor deposition process can be selected in the same ranges as those described for the vacuum vapor deposition of the hole-injecting layer although the conditions are different depending on the used compound.

Next, an electron-injecting layer is formed on the light-emitting layer formed above. Similarly to the hole-injecting layer and the light-emitting layer, it is preferable that the electron-injecting layer is formed in accordance with the vacuum vapor deposition process since a uniform film must be obtained. The conditions of the vacuum vapor deposition can be selected in the same ranges as those described for the vacuum vapor deposition of the hole-injecting layer and the light-emitting layer.

When the vapor deposition process is used, the compound of the present invention can be vapor deposited in combination with other materials although the situation may be different depending on which layer in the light-emitting zone or in the hole-transporting zone comprises the compound. When the spin coating process is used, the compound can be incorporated into the formed layer by using a mixture of the compound with other materials.

A cathode is formed on the electron-injecting layer formed above in the last step, and an organic EL device can be obtained.

The cathode is made of a metal and can be formed in accordance with the vacuum vapor deposition process or the sputtering process. It is preferable that the vacuum vapor deposition process is used in order to prevent formation of damages on the lower organic layers during the formation of the film.

In the above preparation of the organic EL device, it is preferable that the above layers from the anode to the cathode are formed successively while the preparation system is kept in a vacuum after being evacuated once.

The process for forming the layers in the organic EL device of the present invention is not particularly limited. A conventional process such as the vacuum vapor deposition process and the spin coating process can be used. The organic thin film layer which is used in the organic EL device of the present invention and comprises the compound represented by general formula (1) described above can be formed in accordance with a conventional process such as the vacuum vapor deposition process and the molecular beam epitaxy process (the MBE process) or, using a solution prepared by dissolving the compounds into a solvent, in accordance with a coating process such as the dipping process, the spin coating process, the casting process, the bar coating process and the roll coating process.

The thickness of each layer in the organic thin film layer in the organic EL device of the present invention is not particularly limited. In general, an excessively thin layer tends to have defects such as pin holes, and an excessively thick layer requires a high applied voltage to decrease the efficiency. Therefore, a thickness in the range of several nanometers to 1 μm is preferable.

The organic EL device which can be prepared as described above emits light when a direct voltage of 5 to 40V is applied in the condition that the anode is connected to a positive electrode (+) and the cathode is connected to a negative electrode (−). When the connection is reversed, no electric current is observed and no light is emitted at all. When an alternating voltage is applied to the organic EL device, the uniform light emission is observed only in the condition that the polarity of the anode is positive and the polarity of the cathode is negative. When an alternating voltage is applied to the organic EL device, any type of wave shape can be used.

### EXAMPLES

Hereinafter, the present invention will be described in detail on the basis of examples. However, the present invention is not limited to the following examples within the gist of the present invention.

### Synthesis Example 1

### Synthesis of N,N-diphenyl-4-amino-4′-iodo-1,1′-biphenyl

In a stream of argon, 1,058 g of N,N-diphenylamine (manufactured by Tokyo Chemical Industry Co., Ltd.), 2,542 g of 4,4′-diiodobiphenyl (manufactured by Wako Pure Chemical Industries, Ltd.), 1,296 g of potassium carbonate (manufactured by Wako Pure Chemical Industries, Ltd.), 39.8 g of a copper powder (manufactured by Wako Pure Chemical Industries, Ltd.), and 4 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. 3 L of toluene were added to the residue, a precipitated crystal was filtered out and removed, and the filtrate was concentrated. Then, 10 L of methanol were added to the residue, and the whole was stirred. After that, the supernatant was wasted, and 3 L of methanol were additionally added to the remainder. After the mixture had been stirred, the supernatant was wasted, and the remainder was subjected to column purification. As a result, a yellow powder was produced. The powder was dissolved into 1.5 L of toluene under heat, 1.5 L of hexane were added to the solution, and the whole was cooled. A precipitated crystal was filtered out. As a result, 1,343 g of N,N-diphenyl-4-amino-4′-iodo-1,1′-biphenyl were produced.

### Synthesis Example 2

### Synthesis of N-(1-naphthyl)-N-phenyl-4-amino-4′-iodo-1,1′-biphenyl

In a stream of argon, 1,371 g of N-phenyl-1-naphthylamine (manufactured by Kanto Chemical Co., Inc.), 2,542 g of 4,4′-diiodobiphenyl (manufactured by Wako Pure Chemical Industries, Ltd.), 1,296 g of potassium carbonate (manufactured by Wako Pure Chemical Industries, Ltd.), 39.8 g of a copper powder (manufactured by Wako Pure Chemical Industries, Ltd.), and 4 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. 3 L of toluene were added to the residue, a precipitated crystal was filtered out and removed, and the filtrate was concentrated. Then, 10 L of methanol were added to the residue, and the whole was stirred. After that, the supernatant was wasted, and 3 L of methanol were additionally added to the remainder. After the mixture had been stirred, the supernatant was wasted, and the remainder was subjected to column purification. As a result, a yellow powder was produced. The powder was dissolved into 1.5 L of toluene under heat, 1.5 L of hexane were added to the solution, and the whole was cooled. A precipitated crystal was filtered out. As a result, 640 g of N-(1-naphthyl)-N-phenyl-4-amino-4′-iodo-1,1′-biphenyl were produced.

### Synthesis Example 3

### Synthesis of N,N-di(2-naphthyl)-4-amino-4′-iodo-1,1′-biphenyl

In a stream of argon, 1,684 g of N,N-di(2-naphthyl)amine (manufactured by Nihon SiberHegner Co., Ltd.), 2,542 g of 4,4′-diiodobiphenyl (manufactured by Wako Pure Chemical Industries, Ltd.), 1,296 g of potassium carbonate (manufactured by Wako Pure Chemical Industries, Ltd.), 39.8 g of a copper powder (manufactured by Wako Pure Chemical Industries, Ltd.), and 4 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. 3 L of toluene were added to the residue, a precipitated crystal was filtered out and removed, and the filtrate was concentrated. Then, 10 L of methanol were added to the residue, and the whole was stirred. After that, the supernatant was wasted, and 3 L of methanol were additionally added to the remainder. After the mixture had been stirred, the supernatant was wasted, and the remainder was subjected to column purification. As a result, a yellow powder was produced. The powder was dissolved into 1.5 L of toluene under heat, 1.5 L of hexane were added to the solution, and the whole was cooled. A precipitated crystal was filtered out. As a result, 697 g of N,N-di(2-naphthyl)-4-amino-4′-iodo-1,1′-biphenyl were produced.

### Synthesis Example 4

### Synthesis of N,N-di(1-naphthyl)-4,4′-benzidine

In a stream of argon, 547 g of 1-acetamidenaphthalene (manufactured by Tokyo Chemical Industry Co., Ltd.), 400 g of 4,4′-diiodobiphenyl (manufactured by Wako Pure Chemical Industries, Ltd.), 544 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 4 days.

After the reaction, the resultant was cooled, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and 382 g of a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 292 g of N,N′-di(1-naphthyl)-4,4′-benzidine were produced.

### Synthesis Example 5

### Synthesis of N-(1-naphthyl)-N′-phenyl-4,4′-benzidine

In a stream of argon, 182 g of 1-acetamidenaphthalene (manufactured by Tokyo Chemical Industry Co., Ltd.), 400 g of 4,4′-diiodobiphenyl (manufactured by Wako Pure Chemical Industries, Ltd.), 204 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 3 days.

After the reaction, the resultant was cooled, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 264 g of N-(1-naphthyl)-4-amino-4′-iodo-1,1′-biphenyl were produced.

Next, in a stream of argon, 250 g of N-(1-naphthyl)-4-amino-4′-iodo-1,1′-biphenyl, 160 g of acetanilid (manufactured by Wako Pure Chemical Industries, Ltd.), 165 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 4 days.

After the reaction, the resultant was cooled, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 155 g of N-(1-naphthyl)-N′-phenyl-4,4′-benzidine were produced.

### Synthesis Example 6

### Synthesis of N-(1-naphthyl)-N′-(4-phenyl)-4,4′-benzidine

In a stream of argon, 873 g of 4,4′-diiodobiphenyl (manufactured by Tokyo Chemical Industry Co., Ltd.), 398 g of 1-acetamidenaphthalene (manufactured by Tokyo Chemical Industry Co., Ltd.), 600 g of potassium carbonate (manufactured by Tokyo Chemical Industry Co., Ltd.), 20.2 g of copper iodide (manufactured by Wako Pure Chemical Industries, Ltd.), 19.0 g of N,N′-dimethylethylenediamine (manufactured by SIGMA-ALDRICH), and 2.5 L of xylene (manufactured by Wako Pure Chemical Industries, Ltd.) were loaded and refluxed under heat for 3 days.

After the reaction, the resultant was cooled, and insoluble matter was filtered out. The product that had been filtered out was dissolved into 25 L of chloroform, and insoluble matter was removed. After that, the resultant was concentrated. The resultant solid was recrystallized with toluene. As a result, 360 g of N-(1-naphthyl)-N-acetyl-4-amino-4′-iodobiphenyl were produced.

Next, in a stream of argon, 275 g of N-(1-naphthyl)-N-acetyl-4-amino-4′-iodobiphenyl synthesized in advance, 250 g of acetanilide, 165 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 4 days.

After the reaction, the resultant was cooled, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 165 g of N-(1-naphthyl)-N′-(4-phenyl)-4,4′-benzidine were produced.

### Synthesis Example 7

### Synthesis of 2-acetamidenaphthalene

In a stream of argon, 444 g of 2-bromonaphthalene (manufactured by Tokyo Chemical Industry Co., Ltd.), 151 g of acetamide (manufactured by Tokyo Chemical Industry Co., Ltd.), 600 g of potassium carbonate (manufactured by Tokyo Chemical Industry Co., Ltd.), 20.2 g of copper iodide (manufactured by Wako Pure Chemical Industries, Ltd.), 19.0 g of N,N′-dimethylethylenediamine (manufactured by SIGMA-ALDRICH), and 2.5 L of xylene (manufactured by Wako Pure Chemical Industries, Ltd.) were loaded and refluxed under heat for 3 days. After the reaction, the resultant was cooled, and insoluble matter was filtered out. The product that had been filtered out was dissolved into 25 L of chloroform, and insoluble matter was removed. After that, the resultant was concentrated. The resultant solid was recrystallized with toluene. As a result, 340 g of 4-acetamidebiphenyl were produced.

### Synthesis Example 8

### Synthesis of N,N′-di(2-naphthyl)-4,4′-benzidine

In a stream of argon, 547 g of 2-acetamidenaphthalene (manufactured by Tokyo Chemical Industry Co., Ltd.), 400 g of 4,4′-diiodobiphenyl (manufactured by Wako Pure Chemical Industries, Ltd.), 544 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 4 days.

After the reaction, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and 382 g of a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 264 g of N,N′-di(2-naphthyl)-4,4′-benzidine were produced.

### Synthesis Example 9

### Synthesis of N-(2-naphthyl)-N′-phenyl-4,4′-benzidine

In a stream of argon, 182 g of 2-acetamidenaphthalene (manufactured by Tokyo Chemical Industry Co., Ltd.), 400 of 4,4′-diiodobiphenyl (manufactured by Wako Pure Chemical Industries, Ltd.), 204 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 3 days.

After the reaction, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 251 g of N-(2-naphthyl)-4-amino-4′-iodobiphenyl were produced.

Next, in a stream of argon, 250 g of N-(2-naphthyl)-4-amino-4′-iodobiphenyl, 160 g of 1-acetanilid (manufactured by Wako Pure Chemical Industries, Ltd.), 165 g of potassium carbonate (manufactured by Wako Pure Chemical Industries, Ltd.), 12.5 g of a copper powder (manufactured by Wako Pure Chemical Industries, Ltd.), and 2 L of decalin were loaded and allowed to react with one another at 190° C. for 4 days.

After the reaction, the resultant was cooled, 2 L of toluene were added to the resultant, and insoluble matter was filtered out. The product that had been filtered out was dissolved into 4.5 L of chloroform, and insoluble matter was removed. After that, the resultant was treated with activated carbon and concentrated. 3 L of acetone were added to the resultant, and a precipitated crystal were filtered out.

The crystal was suspended into 5 L of ethylene glycol (manufactured by Wako Pure Chemical Industries, Ltd.) and 50 mL of water, and 145 g of an 85% aqueous solution of potassium hydroxide were added to the suspension. After that, the resultant was subjected to a reaction at 120° C. for 2 hours.

After the reaction, the reaction liquid was injected into 10 L of water, and a precipitated crystal was filtered out and washed with water and methanol.

The resultant crystal was dissolved into 3 L of tetrahydrofuran under heat, and the solution was treated with activated carbon and concentrated. Acetone was added to the concentrate to precipitate a crystal. The crystal was filtered out. As a result, 131 g of N-(2-naphthyl)-N′-phenyl-4,4′-benzidine were produced.

### Synthesis Example 10

### Synthesis of N-(2-naphthyl)-N-phenyl-4-amino-4′-iodo-1,1′-biphenyl

In a stream of argon, 1,371 g of N-phenyl-2-naphthylamine (manufactured by Kanto Chemical Co., Inc.), 2,542 g of 4,4′-diiodobiphenyl (manufactured by Wako Pure Chemical Industries, Ltd.), 1,296 g of potassium carbonate (manufactured by Wako Pure Chemical Industries, Ltd.), 39.8 g of a copper powder (manufactured by Wako Pure Chemical Industries, Ltd.), and 4 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. 3 L of toluene were added to the residue, a precipitated crystal was filtered out and removed, and the filtrate was concentrated. Then, 10 L of methanol were added to the residue, and the whole was stirred. After that, the supernatant was wasted, and 3 L of methanol were additionally added to the remainder. After the mixture had been stirred, the supernatant was wasted, and the remainder was subjected to column purification. As a result, a yellow powder was produced. The powder was dissolved into 1.5 L of toluene under heat, 1.5 L of hexane were added to the solution, and the whole was cooled. A precipitated crystal was filtered out. As a result, 590 g of N-(2-naphthyl)-N-phenyl-4-amino-4′-iodo-1,1′-biphenyl were produced.

### Synthesis Example 11

### Synthesis of N-(1-naphthyl)-N-phenyl-4-amino-4′-iodo-1,1′-biphenyl

In a stream of argon, 1,371 g of 1-phenyl-1-naphthylamine (manufactured by Kanto Chemical Co., Inc.), 2,542 g of 4,4′-diiodobiphenyl (manufactured by Wako Pure Chemical Industries, Ltd.), 1,296 g of potassium carbonate (manufactured by Wako Pure Chemical Industries, Ltd.), 39.8 g of a copper powder (manufactured by Wako Pure Chemical Industries, Ltd.), and 4 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. 3 L of toluene were added to the residue, a precipitated crystal was filtered out and removed, and the filtrate was concentrated. Then, 10 L of methanol were added to the residue, and the whole was stirred. After that, the supernatant was wasted, and 3 L of methanol were additionally added to the remainder. After the mixture had been stirred, the supernatant was wasted, and the remainder was subjected to column purification. As a result, a yellow powder was produced. The powder was dissolved into 1.5 L of toluene under heat, 1.5 L of hexane were added to the solution, and the whole was cooled. A precipitated crystal was filtered out. As a result, 540 g of N-(1-naphthyl)-N-phenyl-4-amino-4′-iodo-1,1′-biphenyl were produced.

### Example 1

### Synthesis of TA-2

In a stream of argon, 25 g of N-(1-naphthyl)-N-phenyl-4-amino-4′-iodo-1,1′-biphenyl, 10 g of N,N′-di(1-naphthyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 11 g of a pale yellow powder were obtained.

The powder was identified as TA-2 because a main peak having an m/z of 1,175 was obtained for C₈₈H₆₂N₄=1,174 through field diffusion-mass spectral (FD-MS) analysis.

### Example 2

### Synthesis of TA-6

In a stream of argon, 32 g of N,N-di(2-naphthyl)-4-amino-4′-iodo-1,1′-biphenyl, 10 g of N,N′-di(1-naphthyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 12 g of a pale yellow powder were obtained.

The powder was identified as TA-6 because a main peak having an m/z of 1,275 was obtained for C₉₆H₆₆N₄=1,274 through field diffusion-mass spectral (FD-MS) analysis.

### Example 3

### Synthesis of TA-7

In a stream of argon, 11 g of N,N-diphenyl-4-amino-4′-iodo-1,1′-biphenyl, 10 g of N,N′-di(1-naphthyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 9.3 g of a pale yellow powder were obtained.

The powder was identified as TA-7 because a main peak having an m/z of 1,075 was obtained for C₈₀H₅₈N₄=1,074 through field diffusion-mass spectral (FD-MS) analysis.

### Example 4

### Synthesis of TA-8

In a stream of argon, 11 g of N,N-diphenyl-4-amino-4′-iodo-1,1′-biphenyl, 8.8 g of N-(1-naphthyl)-N′-phenyl-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 10 g of a pale yellow powder were obtained.

The powder was identified as TA-8 because a main peak having an m/z of 1,025 was obtained for C₈₀H₅₈N₄=1,024 through field diffusion-mass spectral (FD-MS) analysis.

### Example 5

### Synthesis of TA-3

In a stream of argon, 32 g of N,N-di(2-naphthyl)-4-amino-4′-iodo-1,1′-biphenyl, 7.7 g of N,N′-diphenyl-4,4′-benzidine (manufactured by Wako Pure Chemical Industries, Ltd.), 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 9.1 g of a pale yellow powder were obtained.

The powder was identified as TA-3 because a main peak having an m/z of 1,175 was obtained for C₈₈H₆₂N₄=1,174 through field diffusion-mass spectral (FD-MS) analysis.

### Example 6

### Synthesis of TA-9

In a stream of argon, 32 g of N,N-di(2-naphthyl)-4-amino-4′-iodo-1,1′-biphenyl, 8.8 g of N-(1-naphthyl)-N′-phenyl-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 9.1 g of a pale yellow powder were obtained.

The powder was identified as TA-9 because a main peak having an m/z of 1,225 was obtained for C₉₂H₆₄N₄=1,224 through field diffusion-mass spectral (FD-MS) analysis.

### Example 7

### Synthesis of TA-13

In a stream of argon, 12 g of N,N-di(2-naphthyl)-4-amino-4′-iodo-1,1′-biphenyl, 7.7 g of N,N′-diphenyl-4,4′-benzidine (manufactured by Wako Pure Chemical Industries, Ltd.), 7 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 4.1 g of a precipitated crystal was filtered out.

Next, in a stream of argon, 4.0 g of the precipitated crystal, 3.0 g of N,N-diphenyl-4-amino-4′-iodo-1,1′-biphenyl, 4 g of potassium carbonate (manufactured by Wako Pure Chemical Industries, Ltd.), 0.2 g of a copper powder (manufactured by Wako Pure Chemical Industries, Ltd.), and 500 mL of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 4.1 g of a precipitated crystal was filtered out.

The crystal was subjected to sublimation purification. As a result, 1.8 g of a pale yellow powder were obtained.

The powder was identified as TA-13 because a main peak having an m/z of 1,075 was obtained for C₈₀H₅₈N₄=1,074 through field diffusion-mass spectral (FD-MS) analysis.

### Example 8

### Synthesis of TA-16

In a stream of argon, 32 g of N,N-di(2-naphthyl)-4-amino-4′-iodo-1,1′-biphenyl, 8.2 g of N,N′-di(p-tolyl)-4,4′-benzidine (manufactured by Nihon SiberHegner Co., Ltd.), 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 12 g of a pale yellow powder were obtained.

The powder was identified as TA-16 because a main peak having an m/z of 1,203 was obtained for C₉₀H₆₆N₄=1,202 through field diffusion-mass spectral (FD-MS) analysis.

### Example 9

### Synthesis of TA-17

In a stream of argon, 11 g of N,N-diphenyl-4-amino-4′-iodo-1,1′-biphenyl, 10.5 g of N-(1-naphthyl)-N′-(4-biphenyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 9.2 g of a pale yellow powder were obtained.

The powder was identified as TA-17 because a main peak having an m/z of 1,101 was obtained for C₈₂H₆₀N₄=1,100 through field diffusion-mass spectral (FD-MS) analysis.

### Example 10

### Synthesis of TA-18

In a stream of argon, 25 g of N-(1-naphthyl)-N-phenyl-4-amino-4′-iodo-1,1′-biphenyl, 10.5 g of N-(1-naphthyl)-N′-(4-biphenyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 8.4 g of a pale yellow powder were obtained.

The powder was identified as TA-18 because a main peak having an m/z of 1,201 was obtained for C₉₀H₆₄N₄=1,200 through field diffusion-mass spectral (FD-MS) analysis.

### Example 11

### Synthesis of TA-19

In a stream of argon, 10.9 g of N-(1-naphthyl)-N-phenyl-4-amino-4′-iodo-1,1′-biphenyl, 7.7 g of N,N′-diphenyl-4,4′-benzidine (manufactured by Wako Pure Chemical Industries, Ltd.), 7 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 3.9 g of a precipitated crystal was filtered out.

Next, in a stream of argon, 4.0 g of the precipitated crystal, 3.0 g of N,N-diphenyl-4-amino-4′-iodo-1,1′-biphenyl, 4 g of potassium carbonate (manufactured by Wako Pure Chemical Industries, Ltd.), 0.2 g of a copper powder (manufactured by Wako Pure Chemical Industries, Ltd.), and 500 mL of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 3.2 g of a precipitated crystal was filtered out.

The crystal was subjected to sublimation purification. As a result, 1.5 g of a pale yellow powder were obtained.

The powder was identified as TA-19 because a main peak having an m/z of 1,025 was obtained for C₇₅H₅₆N₄=1,024 through field diffusion-mass spectral (FD-MS) analysis.

### Example 12

### Synthesis of TB-1

In a stream of argon, 11 g of N,N-diphenyl-4-amino-4′-iodo-1,1′-biphenyl, 10 g of N,N′-di(2-naphthyl)-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 8.6 g of a pale yellow powder were obtained.

The powder was identified as TB-1 because a main peak having an m/z of 1,075 was obtained for C₈₀H₅₈N₄=1,074 through field diffusion-mass spectral (FD-MS) analysis.

### Example 13

### Synthesis of TB-2

In a stream of argon, 11 g of N,N-diphenyl-4-amino-4′-iodo-1,1′-biphenyl, 8.8 g of N-(2-naphthyl)-N′-phenyl-4,4′-benzidine, 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 8 g of a pale yellow powder were obtained.

The powder was identified as TB-2 because a main peak having an m/z of 1,025 was obtained for C₈₀H₅₈N₄=1,024 through field diffusion-mass spectral (FD-MS) analysis.

### Example 14

### Synthesis of TB-3

In a stream of argon, 25 g of N-(2-naphthyl)-N-phenyl-4-amino-4′-iodo-1,1′-biphenyl, 7.7 g of N,N′-diphenyl-4,4′-benzidine (manufactured by Wako Pure Chemical Industries, Ltd.), 10 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. A precipitated crystal was filtered out.

The crystal was subjected to sublimation purification. As a result, 9 g of a pale yellow powder were obtained.

The powder was identified as TB-3 because a main peak having an m/z of 1,075 was obtained for C₈₀H₅₈N₄=1,074 through field diffusion-mass spectral (FD-MS) analysis.

### Example 15

### Synthesis of TB-4

In a stream of argon, 10.9 g of N-(2-naphthyl)-N-phenyl-4-amino-4′-iodo-1,1′-biphenyl, 7.7 g of N,N′-diphenyl-4,4′-benzidine (manufactured by Wako Pure Chemical Industries, Ltd.), 7 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 4.2 g of a precipitated crystal was filtered out.

Next, in a stream of argon, 4.0 g of the precipitated crystal, 3.0 g of N,N-diphenyl-4-amino-4′-iodo-1,1′-biphenyl, 4 g of potassium carbonate (manufactured by Wako Pure Chemical Industries, Ltd.), 0.2 g of a copper powder (manufactured by Wako Pure Chemical Industries, Ltd.), and 500 mL of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 3.2 g of a precipitated crystal was filtered out.

The crystal was subjected to sublimation purification. As a result, 1.7 g of a pale yellow powder were obtained.

The powder was identified as TB-4 because a main peak having an m/z of 1,025 was obtained for C₇₆H₅₆N₄=1,024 through field diffusion-mass spectral (FD-MS) analysis.

### Example 16

### Synthesis of TB-19

In a stream of argon, 10.9 g of N-(1-naphthyl)-N-phenyl-4-amino-4′-iodo-1,1′-biphenyl, 7.7 g of N,N′-diphenyl-4,4′-benzidine (manufactured by Wako Pure Chemical Industries, Ltd.), 4 g of potassium carbonate (manufactured by Wako Pure Chemical Industries, Ltd.), 0.4 g of a copper powder (manufactured by Wako Pure Chemical Industries, Ltd.), and 1 L of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 4.6 g of a precipitated crystal was filtered out.

Next, in a stream of argon, 4.0 g of the precipitated crystal, 3.0 g of N,N-diphenyl-4-amino-4′-iodo-1,1′-biphenyl, 4 g of potassium carbonate (manufactured by Wako Pure Chemical Industries, Ltd.), 0.2 g of a copper powder (manufactured by Wako Pure Chemical Industries, Ltd.), and 500 mL of decalin (manufactured by Wako Pure Chemical Industries, Ltd.) were loaded and allowed to react with one another at 200° C. for 6 days.

After the reaction, the resultant was filtered during a hot state. Insoluble matter was washed with toluene, and was concentrated together with the filtrate. Toluene was added to the residue, a precipitated crystal was filtered out and removed, and the filtrate was concentrated. Then, methanol was added to the residue, and the whole was stirred. After that, the supernatant was wasted, and methanol was additionally added to the remainder. After the mixture had been stirred, the supernatant was wasted, and the remainder was subjected to column purification. As a result, a yellow powder was produced. The powder was dissolved into toluene under heat, hexane was added to the solution, and the whole was cooled. 2.7 g of a precipitated crystal was filtered out.

The crystal was subjected to sublimation purification. As a result, 1.3 g of a pale yellow powder were obtained.

The powder was identified as TA-19 because a main peak having an m/z of 1,025 was obtained for C₇₆H₅₆N₄=1,024 through field diffusion-mass spectral (FD-MS) analysis.

### Example 17

### Evaluation of TA-2

A glass substrate with an ITO transparent electrode measuring 25 mm long by 75 mm wide by 1.1 mm thick (manufactured by GEOMATEC Co., Ltd.) was subjected to ultrasonic cleaning in isopropyl alcohol for 5 minutes. After that, the substrate was subjected to UV ozone cleaning for 30 minutes.

The glass substrate with a transparent electrode line after the washing was mounted on a substrate holder of a vacuum vapor deposition device. At first, a TA-2 layer having a thickness of 80 nm was formed on a surface on a side where the transparent electrode line was formed to cover the transparent electrode. The TA-2 layer functions as a hole-transporting layer. Deposition was performed at 1 Å/sec, and a boat temperature at that time was 345 to 350° C.

Furthermore, EMl was deposited from the vapor and formed into a layer having a thickness of 40 nm. At the same time, the following amine compound D1 having a styryl group to serve as a light-emitting molecule was deposited from the vapor in such a manner that a weight ratio between EM1 and D1 would be 40:2. The layer functions as a light-emitting layer.

An Alq layer having a thickness of 10 nm was formed on the layer. The layer functions as an electron-injecting layer. After that, Li serving as a reductive dopant (Li source: manufactured by SAES Getters) and Alq were subjected to co-deposition. Thus, an Alq:Li layer (having a thickness of 10 nm) was formed as an electron-injecting layer (cathode). Metal Al was deposited from the vapor onto the Alq:Li layer to form a metal cathode. As a result, an organic EL light-emitting device was formed.

Table 1 shows the result of the measurements of the half life of light emission in DC constant current driving at an initial luminance of 5,000 cd/m² and room temperature.

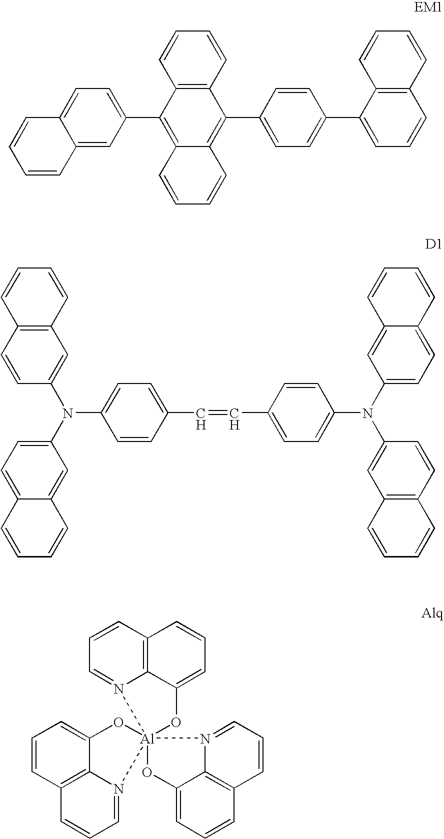

### Example 18

### Evaluation of TA-3

An organic EL light-emitting device was formed in exactly the same manner as in Example 17 except that TA-3 was formed into a layer instead of TA-2. Deposition was performed at 1 Å/sec, and a boat temperature at that time was 336 to 340° C.

Table 1 shows the result of the measurements of the half life of light emission in DC constant current driving at an initial luminance of 5,000 cd/m² and room temperature.

### Example 19

### Evaluation of TA-6

An organic EL light-emitting device was formed in exactly the same manner as in Example 17 except that TA-6 was formed into a layer instead of TA-2. Deposition was performed at 1 Å/sec, and a boat temperature at that time was 339 to 343° C.

Table 1 shows the result of the measurements of the half life of light emission in DC constant current driving at an initial luminance of 5,000 cd/m² and room temperature.

### Example 20

### Evaluation of TA-7

An organic EL light-emitting device was formed in exactly the same manner as in Example 17 except that TA-7 was formed into a layer instead of TA-2. Deposition was performed at 1 Å/sec, and a boat temperature at that time was 314 to 319° C.

Table 1 shows the result of the measurements of the half life of light emission in DC constant current driving at an initial luminance of 5,000 cd/m² and room temperature.

### Example 21

### Evaluation of TA-8

An organic EL light-emitting device was formed in exactly the same manner as in Example 17 except that TA-8 was formed into a layer instead of TA-2. Deposition was performed at 1 Å/sec, and a boat temperature at that time was 310 to 314° C.

Table 1 shows the result of the measurements of the half life of light emission in DC constant current driving at an initial luminance of 5,000 cd/m² and room temperature.

### Example 22

### Evaluation of TA-9

An organic EL light-emitting device was formed in exactly the same manner as in Example 17 except that TA-9 was formed into a layer instead of TA-2. Deposition was performed at 1 Å/sec, and a boat temperature at that time was 326 to 330° C.

Table 1 shows the result of the measurements of the half life of light emission in DC constant current driving at an initial luminance of 5,000 cd/m² and room temperature.

### Example 23

### Evaluation of TA-13

An organic EL light-emitting device was formed in exactly the same manner as in Example 17 except that TA-13 was formed into a layer instead of TA-2. Deposition was performed at 1 Å/sec, and a boat temperature at that time was 321 to 326° C.

Table 1 shows the result of the measurements of the half life of light emission in DC constant current driving at an initial luminance of 5,000 cd/m² and room temperature.

### Example 24

### Evaluation of TA-16

An organic EL light-emitting device was formed in exactly the same manner as in Example 17 except that TA-16 was formed into a layer instead of TA-2. Deposition was performed at 1 Å/sec, and a boat temperature at that time was 343 to 348° C.

Table 1 shows the result of the measurements of the half life of light emission in DC constant current driving at an initial luminance of 5,000 cd/m² and room temperature.

### Example 25

### Evaluation of TA-17

An organic EL light-emitting device was formed in exactly the same manner as in Example 17 except that TA-17 was formed into a layer instead of TA-2. Deposition was performed at 1 Å/sec, and a boat temperature at that time was 322 to 327° C.

Table 1 shows the result of the measurements of the half life of light emission in DC constant current driving at an initial luminance of 5,000 cd/m² and room temperature.

### Example 26

### Evaluation of TA-18

An organic EL light-emitting device was formed in exactly the same manner as in Example 17 except that TA-18 was formed into a layer instead of TA-2. Deposition was performed at 1 Å/sec, and a boat temperature at that time was 338 to 343° C.

Table 1 shows the result of the measurements of the half life of light emission in DC constant current driving at an initial luminance of 5,000 cd/m² and room temperature.

### Example 27

### Evaluation of TA-19

An organic EL light-emitting device was formed in exactly the same manner as in Example 17 except that TA-19 was formed into a layer instead of TA-2. Deposition was performed at 1 521 /sec, and a boat temperature at that time was 341 to 343° C.

Table 1 shows the result of the measurements of the half life of light emission in DC constant current driving at an initial luminance of 5,000 cd/m² and room temperature.

### Comparative Example 1

### Evaluation of ta-1

An organic EL light-emitting device was formed in exactly the same manner as in Example 17 except that ta-1 was formed into a layer instead of TA-2. Deposition was performed at 1 Å/sec, and a boat temperature at that time was 309 to 311° C.

Table 1 shows the result of the measurements of the half life of light emission in DC constant current driving at an initial luminance of 5,000 cd/m² and room temperature.

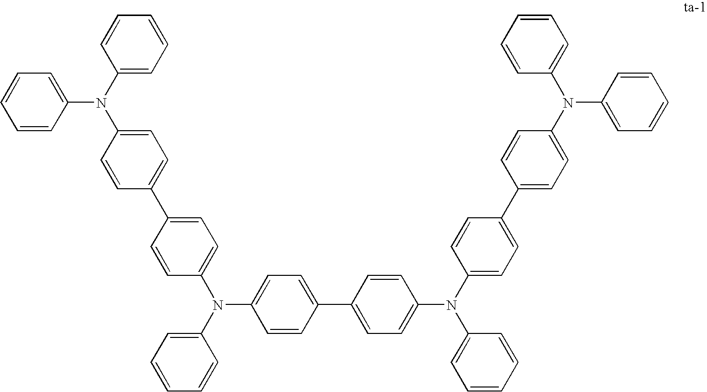

### Comparative Example 2

### Evaluation of ta-2

An organic EL light-emitting device was formed in exactly the same manner as in Example 17 except that ta-2 was formed into a layer instead of TA-2. Deposition was performed at 1 Å/sec, and a boat temperature at that time was 351 to 356° C.

Table 1 shows the result of the measurements of the half life of light emission in DC constant current driving at an initial luminance of 5,000 cd/m² and room temperature.

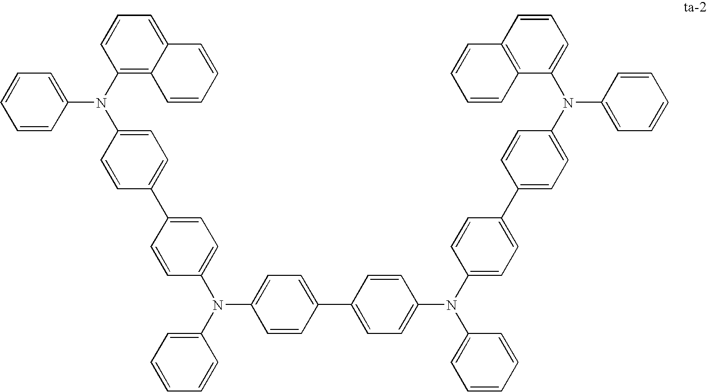

TABLE 1
Table 1

| | | Half life | | | |
|---|---|---|---|---|---|
| | Hole- | of initial | | 1% weight | |
| | trans- | luminance | Evaporation | reduc- | Lumi- |
| | porting | of 5,000 | temperature | tion | nescent |
| | material | cd/m² (h) | (° C.) | (° C.) | color |
| Example 17 | TA-2 | 330 | 345 to 350 | 511 | Blue |
| Example 18 | TA-3 | 320 | 336 to 340 | 501 | Blue |
| Example 19 | TA-6 | 300 | 339 to 343 | 506 | Blue |
| Example 20 | TA-7 | 430 | 314 to 319 | 494 | Blue |
| Example 21 | TA-8 | 480 | 310 to 314 | 492 | Blue |
| Example 22 | TA-9 | 410 | 326 to 330 | 500 | Blue |
| Example 23 | TA-13 | 420 | 321 to 326 | 503 | Blue |
| Example 24 | TA-16 | 300 | 343 to 348 | 509 | Blue |
| Example 25 | TA-17 | 410 | 322 to 327 | 501 | Blue |
| Example 26 | TA-18 | 400 | 338 to 343 | 501 | Blue |
| Example 27 | TA-19 | 480 | 341 to 343 | 512 | Blue |
| Comparative | ta-1 | 120 | 309 to 311 | 498 | Blue |
| example 1 | | | | | |
| Comparative | ta-2 | 180 | 351 to 356 | 508 | Blue |
| example 2 | | | | | |

* The term “1% weight reduction” in the table refers to the temperature at which the weight of a sample reduces by 1% as compared to the weight at the initiation of measurement in thermogravimetric analysis in a stream of nitrogen at a constant rate of temperature increase.

As can be seen from the above results, when the amine derivative of the present invention was used as a hole-transporting material for an organic EL device, the attenuation of emission luminance was smaller than that in the case of a tetramer amine derivative that had been conventionally used. In particular, in a blue light-emitting device, a reducing effect on the attenuation was significant.

One possible reason for the attenuation of the emission luminance of organic EL is the injection of an excessive electron not involved in recombination into a hole-transporting layer to cause some degradation like Comparative Example 1. A tetramer amine having a fused ring in its molecule has some stability with respect to the injection of an electron. The inventors consider that at least two of Ar¹ to Ar⁶ suitably each represent a substituted or unsubstituted fused aromatic ring having 10 to 20 nuclear carbon atoms.

On the other hand, when the number of fused rings is large, the evaporation temperature of a tetramer amine tends to increase. When the evaporation temperature excessively increases so that a difference between the evaporation temperature and the decomposition temperature of a molecule becomes small, the attenuation of emission luminance tends to be abrupt when the tetramer amine is used in an organic EL device like Comparative Example 2. As long as a fused ring is present at the position of Ar³ and/or Ar⁴, sufficient durability against an electron is obtained even when the number of fused rings is one. As a result, the attenuation of emission luminance is suppressed. In addition, when a fused ring is introduced into the position of Ar³ and/or Ar⁴, an increase in evaporation temperature at such position is relatively small as compared to that at any other site, so heat decomposition at the time of evaporation may be suppressed.

Furthermore, a tetramer amine having lower symmetric property tends to have a lower evaporation temperature. As a result, a light emission life is additionally improved.

### Example 28

A glass substrate with an ITO transparent electrode measuring 25 mm long by 75 mm wide by 1.1 mm thick (manufactured by GEOMATEC Co., Ltd.) was subjected to ultrasonic cleaning in isopropyl alcohol for 5 minutes. After that, the substrate was subjected to UV ozone cleaning for 30 minutes.

The glass substrate with a transparent electrode line after the washing was mounted on a substrate holder of a vacuum vapor deposition device. At first, a TB-1 layer having a thickness of 60 nm was formed on a surface on a side where the transparent electrode line was formed to cover the transparent electrode. The TB-1 layer functions as a hole-transporting layer. The change of the degree of vacuum upon formation of the TB layer was monitored by the vacuum measure.

An HT1 layer having a thickness of 20 nm was formed on the TB-1 layer subsequently to the formation of the TB-1 layer. The layer functions as a hole-transporting layer.

Furthermore, EM1 was deposited from the vapor and formed into a layer having a thickness of 40 nm. At the same time, the following amine compound D1 having a styryl group to serve as a light-emitting molecule was deposited from the vapor in such a manner that a weight ratio between EM1 and D1 would be 40:2. The layer functions as a light-emitting layer.

An Alq layer having a thickness of 20 nm was formed on the layer. The layer functions as an electron-injecting layer. After that, lithium fluoride having a thickness of 1 nm was deposited from the vapor. Metal Al was deposited from the vapor onto the lithium fluoride layer to form a metal cathode. As a result, an organic EL light-emitting device was formed.

Table 2 shows the result of the measurements of the half life of light emission in DC constant current driving at an initial luminance of 1,000 cd/m² and room temperature, and the degree of vacuum upon formation of the TB-1 layer.

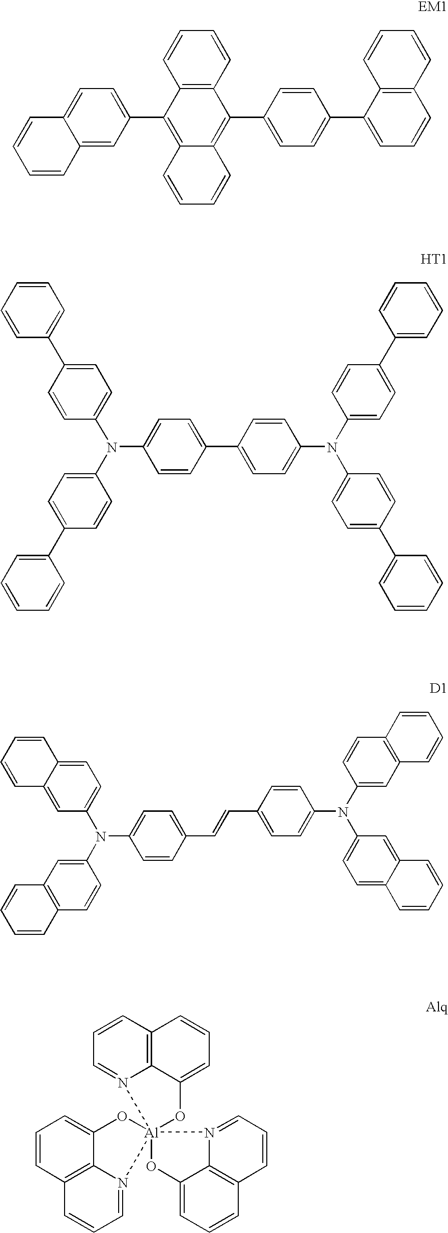

### Example 29

An organic EL light-emitting device was formed in exactly the same manner as in Example 28 except that TB-2 was formed into a layer instead of TB-1.

Table 2 shows the result of the measurements of the half life of light emission in DC constant current driving at an initial luminance of 1,000 cd/m² and room temperature, and the degree of vacuum upon formation of the TB-2 layer.

### Example 30

An organic EL light-emitting device was formed in exactly the same manner as in Example 28 except that TB-3 was formed into a layer instead of TB-1.

Table 2 shows the result of the measurements of the half life of light emission in DC constant current driving at an initial luminance of 1,000 cd/m² and room temperature, and the degree of vacuum upon formation of the TB-3 layer.

### Example 31

An organic EL light-emitting device was formed in exactly the same manner as in Example 28 except that TB-4 was formed into a layer instead of TB-1.

Table 2 shows the result of the measurements of the half life of light emission in DC constant current driving at an initial luminance of 1,000 cd/m² and room temperature, and the degree of vacuum upon formation of the TB-4 layer.

### Example 32

An organic EL light-emitting device was formed in exactly the same manner as in Example 28 except that TB-19 was formed into a layer instead of TB-1.

Table 2 shows the result of the measurements of the half life of light emission in DC constant current driving at an initial luminance of 1,000 cd/m² and room temperature, and the degree of vacuum upon formation of the TB-19 layer.

### Comparative Example 3

An organic EL light-emitting device was formed in exactly the same manner as in Example 28 except that the compound A was formed into a layer instead of TB-1.

Table 2 shows the result of the measurements of the half life of light emission in DC constant current driving at an initial luminance of 1,000 cd/m² and room temperature, and the degree of vacuum upon formation of the compound A layer.

### Comparative Example 4

An organic EL light-emitting device was formed in exactly the same manner as in Example 28 except that the compound B was formed into a layer instead of TB-1.

Table 2 shows the result of the measurements of the half life of light emission in DC constant current driving at an initial luminance of 1,000 cd/m² and room temperature, and the degree of vacuum upon formation of the compound B layer.

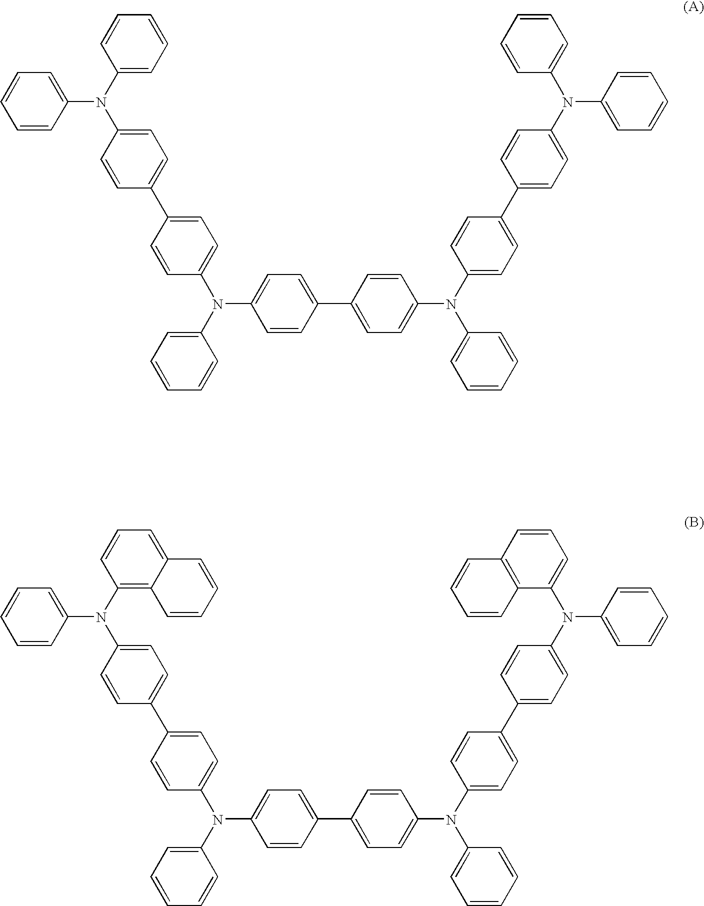

TABLE 2
Table 2

| | | | Evapora- | Degree of | |
|---|---|---|---|---|---|
| | Hole- | | tion | vacuum at | |
| | trans- | Half | tempera- | the time of | Lumi- |
| | porting | life | ture | evaporation | nescent |
| | material | (hour) | (° C.) | (Pa) | color |
| Example 28 | TB-1 | 10000 | 440 to 450 | 1 × 10⁻⁴ | Blue |
| Example 29 | TB-2 | 8000 | 440 to 450 | 1 × 10⁻⁴ | Blue |
| Example 30 | TB-3 | 9000 | 440 to 450 | 1 × 10⁻⁴ | Blue |
| Example 31 | TB-4 | 8000 | 440 to 450 | 1 × 10⁻⁴ | Blue |
| Example 32 | TB-19 | 6500 | 440 to 450 | 2 × 10⁻⁴ | Blue |
| Comparative | Compound | 5000 | 440 to 450 | 3 × 10⁻⁴ | Blue |
| example 3 | A | | | | |
| Comparative | Compound | 5000 | 440 to 450 | 4 × 10⁻⁴ | Blue |
| example 4 | B | | | | |

As described above, the compound B having 1-naphthyl introduced thereinto or the compound A having a phenyl group decomposed to deteriorate the degree of vacuum. In contrast, none of the compounds TB-1 to TB-4 of the present invention each having a 2-naphthyl group introduced thereinto did not show a reduction in degree of vacuum in association with decomposition even at a high temperature. As a result, each of the compounds had a longer lifetime than that of a tetramer amine derivative that had been conventionally used. A prolonging effect on a lifetime was particularly significant in TB-1 in which a 2-naphthyl group was introduced into each of both Ar³ and Ar⁴ and TB-3 in which a 2-naphthyl group was introduced into each of both Ar¹ and Ar⁵.

As shown in the following view, the site of the typical tetramer amine compound A having high reactivity is a para position with respect to nitrogen binding to a terminal phenyl group. The site may react with an adjacent molecule, an oxygen molecule, or the like upon heating at a high temperature to cause heat decomposition.

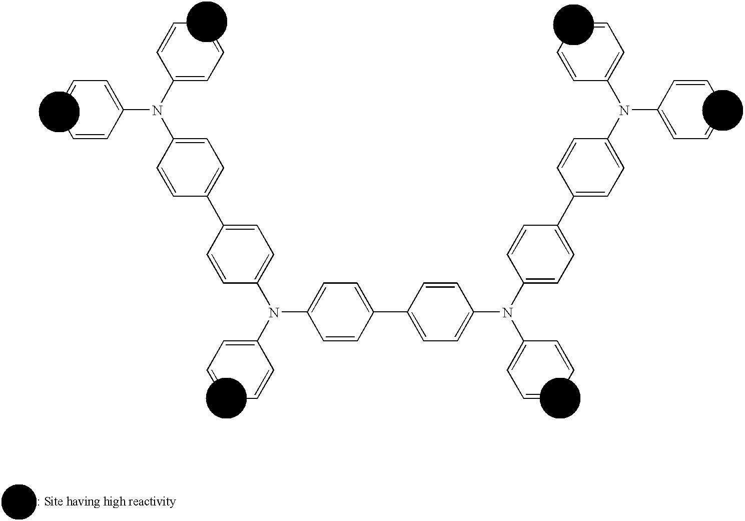

The compound of the present invention into which a substituted or unsubstituted 2-naphthyl group is introduced is of a structure for protecting the site having high reactivity (para position with respect to N at a terminal phenyl group), and is of a structure for delocalizing the charge density of the site having high reactivity, so the reactivity of a molecule reduces. As a result, the heat stability of the molecule is specifically high. Therefore, the compound of the present invention into which a substituted or unsubstituted 2-naphthyl group is introduced can be stably deposited from the vapor even at a high temperature as compared to a compound into which a 1-naphthyl group or a phenyl group is introduced, so a blue organic EL device having a long lifetime can be realized.

### INDUSTRIAL APPLICABILITY

As described above in detail, an organic EL device using the aromatic amine compound of the present invention shows various luminescent color tones and has high heat resistance. In particular, when the aromatic amine compound of the present invention is used as a hole-injecting or -transporting material, hole-injecting or -transporting property is high, and high emission luminance, high emission efficiency, and along lifetime can be obtained. Therefore, the organic EL device of the present invention has high practicability, and is useful for the flat luminous element of a wall hanging television or for a light source such as the backlight of a display. The compound of the present invention can be used for an organic EL device, a hole-injecting or -transporting material, or a charge-transporting material for an electrophotographic photosensitive member or an organic semiconductor.

1. An aromatic amine derivative represented by the following general formula (I), 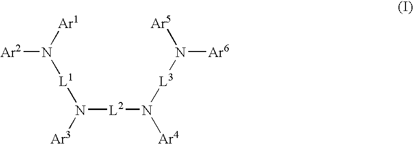
where:
Ar¹ to Ar⁶ each independently represent a substituted or unsubstituted aryl group having 6 to 20 nuclear atoms;
L¹ to L³ each independently represent a linking group represented by the following general formula (II), 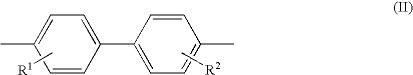
where R¹ and R² each independently represent a hydrogen atom, a substituted or unsubstituted alkyl group having 1 to 6 carbon atoms, or a substituted or unsubstituted aryl group having 6 to 20 nuclear carbon atoms, and R¹ and R² may be coupled with each other to form a saturated or unsaturated ring; and
Ar¹ to Ar⁶ satisfy one of the following conditions (a) to (c) (a) at least two of Ar¹ to Ar³ each represent a substituted or unsubstituted fused aromatic ring having 10 to 20 nuclear carbon atoms;
(b) at least one of Ar³ and Ar⁴ represents a substituted or unsubstituted fused aromatic ring having 10 to 20 nuclear carbon atoms;
(c) only one of Ar¹, Ar², Ar⁵, and Ar⁶ represents a substituted or unsubstituted fused aromatic ring having 10 to 20 nuclear carbon atoms.

2. An aromatic amine derivative according to claim 1, wherein at least two of Ar¹ to Ar³ in the general formula (I) each represent a substituted or unsubstituted fused aromatic ring having 10 to 20 nuclear carbon atoms.

3. An aromatic amine derivative according to claim 1, wherein at least one of Ar³ and Ar⁴ in the general formula (I) represents a substituted or unsubstituted fused aromatic ring having 10 to 20 nuclear carbon atoms.

4. An aromatic amine derivative according to claim 1, wherein only one of Ar¹, Ar², Ar⁵, and Ar⁶ represents a substituted or unsubstituted fused aromatic ring having 10 to 20 nuclear carbon atoms.

5. An aromatic amine derivative represented by the following general formula (I′), 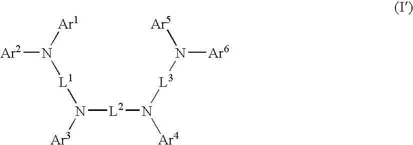
where:
Ar¹ to Ar⁶ each independently represent a substituted or unsubstituted aryl group having 6 to 20 nuclear atoms;
at least one of Ar¹ to Ar⁶ represents a substituted or unsubstituted 2-naphthyl group; and
L¹ to L³ each independently represent a linking group represented by the following general formula (II′), 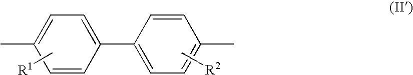
where R¹ and R² each independently represent a hydrogen atom, a substituted or unsubstituted alkyl group having 1 to 6 carbon atoms, or a substituted or unsubstituted aryl group having 6 to 20 nuclear carbon atoms, and R¹ and R² may be coupled with each other to form a saturated or unsaturated ring.

6. An aromatic amine derivative according to claim 5, wherein at least one of Ar³ an Ar⁴ in the general formula (I′) represents a substituted or unsubstituted 2-naphthyl group.

7. An aromatic amine derivative according to claim 5, wherein at least one of Ar¹ and Ar⁵ in the general formula (I′) represents a substituted or unsubstituted 2-naphthyl group.

8. An aromatic amine derivative according to claim 5 or 6, wherein Ar³ and Ar⁴ in the general formula (I′) each represent a substituted or unsubstituted 2-naphthyl group.

9. An aromatic amine derivative according to claim 5 or 7, wherein Ar¹ and Ar⁵ in the general formula (I′) each represent a substituted or unsubstituted 2-naphthyl group.

10. An aromatic amine derivative according to claim 9, wherein Ar² to Ar⁴ and Ar⁶ in the general formula (I′) each independently represent a substituted or unsubstituted aryl group having 6 to 20 nuclear atoms.

11. An aromatic amine derivative according to any one of claims 1 to 10, wherein L¹ to L³ in the general formulae (I) and (I′) are each independently selected from the group consisting of the following general formulae (III-1) to (III-4), 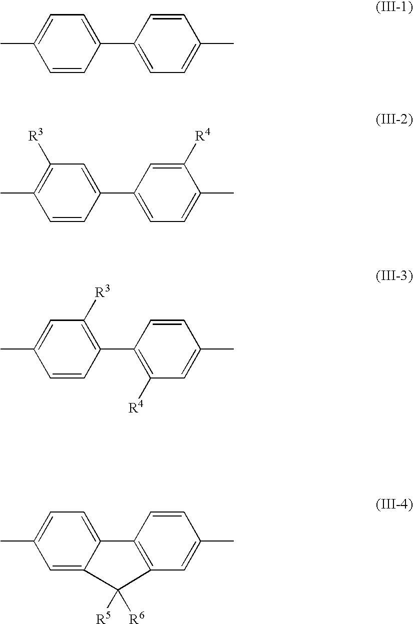
where R³ to R⁶ each independently represent a substituted or unsubstituted alkyl group having 1 to 6carbon atoms, or a substituted or unsubstituted aryl group having 6 to 20 nuclear carbon atoms, and R⁵ and R⁶ may be coupled with each other to form a saturated or unsaturated ring.

12. An aromatic amine derivative according to any one of claims 1 to 11, which comprises a material for organic electroluminescence.

13. An organic electroluminescent device comprising:
a cathode;
an anode; and
one or multiple organic thin film layers having at least a light-emitting layer, the one or multiple organic thin film layers being interposed between the cathode and the anode,
wherein at least one layer of the one or multiple organic thin film layers contains the aromatic amine derivative according to any one of claims 1 to 11 alone or as a component of a mixture.

14. An organic electroluminescent device according to claim 13, wherein:
the one or multiple organic thin film layers have a hole-transporting zone and/or a hole-injecting zone; and
the aromatic amine derivative is incorporated into the hole-transporting zone and/or the hole-injecting zone alone or as a component of a mixture.

15. An organic electroluminescent device according to claim 13, wherein:
the one or multiple organic thin film layers have a hole-transporting layer and/or a hole-injecting layer; and
the aromatic amine derivative is incorporated into the hole-transporting layer and/or the hole-injecting layer.

16. An organic electroluminescent device according to claim 15, wherein the hole-transporting layer and/or the hole-injecting layer mainly contain/contains the aromatic amine derivative.

17. An organic electroluminescent device according to claim 13, wherein the layer containing the aromatic amine derivative contacts with the anode.

18. An organic electroluminescent device according to claim 17, wherein the layer contacting with the anode is mainly composed of the aromatic amine derivative.

19. An organic electroluminescent device according to claim 13, wherein the one or multiple organic thin film layers have a layer containing the aromatic amine derivative and a light-emitting material.

20. An organic electroluminescent device according to claim 13, wherein the one or multiple organic thin film layers have a laminate of a hole-transporting layer and/or a hole-injecting layer containing the aromatic amine derivative and a light-emitting layer composed of a phosphorescent metal complex and a host material.

21. An organic electroluminescent device according to any one of claims 13 to 20, which emits blue-based light.
